(19) United States
(12) Patent Application Publication
(10) Pub. No.: US 2013/0143806 A1
(43) Pub. Date: Jun. 6, 2013

(54) METHOD FOR EARLY PROGNOSIS OF KIDNEY DISEASE

(71) Applicant: Gary Nelsestuen, St. Paul, MN (US)

(72) Inventor: Gary Nelsestuen, St. Paul, MN (US)

(73) Assignee: Regents of the University of Minnesota, St. Paul, MN (US)

(21) Appl. No.: 13/641,858

(22) PCT Filed: Apr. 21, 2011
(86) PCT No.: PCT/US2011/033465

§ 371 Date: Feb. 5, 2013

Related U.S. Application Data
(60) Provisional application No. 61/326,505, filed on Apr. 21, 2010.

(51) Int. Cl. G01N 33/68

(52) U.S. Cl. CPC G01N 33/6893
USPC 514/7.7; 435/7.92; 435/23; 514/167; 600/1

(57) ABSTRACT

Certain embodiments of the present invention relate to methods for detecting kidney disease, in particular early stage kidney disease.

### RELATED APPLICATIONS/PATENTS

The application claims the benefit of priority to U.S. Provisional Patent Application Ser. No. 61/326,505, filed Apr. 21, 2010, which is hereby incorporated by reference in its entirety.

### FIELD OF THE INVENTION

Methods for detecting disease, including kidney disease.

### BACKGROUND OF THE INVENTION

Kidney disease is characterized by slow progression with many years between detection and development of end stage kidney disease. Increased obesity and diabetes have led to increased kidney disease and a substantial increase in the need for kidney transplant. This in turn has produced a great shortage of organ donors. Detection of kidney disease at an early stage is an important goal that can allow time to implement intervention to delay or prevent onset or later stages of kidney disease. Current methods for detection of early stage kidney disease focus on detection of albumin in the urine. For example, current methods for detection of early stage kidney disease depend on several measures such as albumin excretion rate (AER) or albumin to creatinine ratio (ACR). Simple albumin levels in spot urine are unreliable due to the variable concentration of urine. Therefore, AER is often used but requires timed collection of urine and is associated with substantial logistical and compliance problems. Albumin levels may also vary considerably among the healthy population. ACR attempts to correct for urine concentration and allow determination of albumin excretion by normalizing urinary albumin to creatinine, a universal component of urine. However, creatinine excretion varies with individuals for a number of reasons. For example, it arises from muscle tissue so that heavily muscled individuals will excrete higher levels of creatinine than lightly muscled individuals. The ACR varies widely among healthy individuals and requires a substantial change to reach a level that clearly differs from healthy controls.

### SUMMARY OF THE INVENTION

Based on the assays/methods provided herein, a prognosis of future kidney disease as defined by proteinuria is capable at much earlier times than currently available clinical tests. In one embodiment, the methods disclosed herein utilize protein levels relative to uromodulin (ratio) present in urine.

One embodiment provides a method for diagnosing kidney disease from a urine sample comprising determining the ratio of a first protein to a second protein present in said urine sample, wherein the ratio indicates the presence of kidney disease in the sample donor.

Another embodiment provides a method for screening a subject at risk for developing kidney disease comprising determining the ratio of a first protein to a second protein present in urine from said subject, wherein the ratio indicates that the subject is at risk for developing kidney disease.

Another embodiment provides a method for identifying and treating kidney disease in a subject comprising determining the ratio of a first protein to a second protein present in urine from said subject, wherein the ratio indicates the subject has kidney disease, and administering a treatment for kidney disease to the subject. In one embodiment, the treatment comprises surgery, chemotherapy, radiation therapy, dietary restrictions, treatment of high blood pressure (for example, with angiotensin converting enzyme inhibitors (ACEIs) or angiotensin II receptor antagonists), treatment of diabetes, weight management, smoking cessation, treatment of high cholesterol and/or other lipid levels, kidney transplant, dialysis, administration of erythropoietin and/or calcitriol, diuretics, vitamin D, or phosphate binder or a combination thereof. In one embodiment, the subject is administered bardoxolone methyl, olmesartan medoxomil, sulodexide, and avosentan. The method can also contribute to prognosis of coronary artery disease.

One embodiment provides a method for determining whether a subject has kidney disease comprising determining the ratio of a first protein to a second protein present in urine from said subject, wherein the ratio indicates that the subject has developed kidney disease.

In one embodiment, the sample is obtained from a subject at risk for developing kidney disease. In another embodiment, the subject is at risk for developing kidney disease, for example, the subject has a history of diabetes, hypertension (high blood pressure), obesity, sickle cell disease, lupus erythematosus, atherosclerosis, glomerulonephritis, bladder outlet obstruction, overexposure to toxins (e.g., lead) and to some medications (e.g., analgesics), a family history of kidney disease including polycystic kidney disease, is over the age of 60 and/or is a member of one of the following ethnic groups American Indian, African American, Hispanic, Asian American, or Pacific Islander.

In one embodiment the second protein is uromodulin (also known as Tamm-Horsfall Protein). In another embodiment, the first protein is selected from the group consisting of albumin, transferrin, alpha-2-glycoprotein-Zinc, orosomucoid, or leucine-rich alpha-2-glycoprotein.

In one embodiment, the ratios of the first protein to the second protein that are characteristic of early stage kidney disease are at least about one standard deviation above the average for a control, such as a control population (based on a number of healthy individuals), including at least about 2 standard deviations above the average for a control population, such as more than about 2 standard deviations above the average for a control population. In another embodiment, the ratio for albumin to uromodulin is at least about 0.30 (w/w), such as greater than about 0.30 (w/w).

In one embodiment, the ratios of the first protein to uromodulin that are characteristic of early stage kidney disease are more than about 2 standard deviations above the average for a control population. In another embodiment, the ratio for albumin to uromodulin is greater than about 0.30 (w/w).

In one embodiment, the urine protein ratio is combined with at least one other indicator of kidney disease to diagnose development of kidney disease, including, but not limited to, fasting blood glucose, glucose tolerance test outcome, hemoglobin A1C levels, or blood pressure.

In one embodiment, the urine proteins are detected by antibody-based assay (e.g., ELISA). In one embodiment, the antibodies are directed to intact protein. In another embodiment, the urine proteins have been digested with a protease to yield peptides. In one embodiment, the peptides are detected by antibody methods. In another embodiment, the peptides are detected by mass spectrometry methods.

Another embodiment provides a method for detection of disease comprising determining the glycosylation state of urinary peptides, wherein the method comprises measuring the amount of the non-glycosylated form of a putative glycosylated peptide and comparing that to the amount of a peptide of the same protein that is not a target for glycosylation, wherein greater or lesser levels of the unglycosylated peptides compared to a healthy control indicates disease. In one embodiment, the origin of the protein is liver and the disease diagnosis is liver disease. In another embodiment, the origin of the protein is kidney and the disease diagnosis is kidney disease.

### BRIEF DESCRIPTION OF THE DRAWINGS

FIG. 1 depicts protein ratios from spike-in experiments. Spike-in to the standard sample for albumin (solid diamonds) and uromodulin (solid squares). Error bars present the 95% confidence limit as defined by error factor. All spike-in samples are expressed relative to the standard without protein added (zero added protein). Added protein is expressed relative to urinary protein concentration as determined by the BioRad assay. Bias factor was applied. The slope for albumin spike-in (6.65) indicated 0.150 fraction of albumin in the sample. The slope for uromodulin (1.05) indicated 0.95 fraction of total protein as determined by BioRad assay.

FIGS. 2A-C depict relative vs. absolute (w/w) protein ratios for the spike-in experiments. Panel A. Albumin/uromodulin ratio as a function of added uromodulin. The unspiked sample appeared at a ratio of 1.0. Panel B. Relative transferrin to uromodulin ratio as a function of added uromodulin as in panel A. Panel C. Absolute ratio (w/w) of albumin/uromodulin for both the albumin and urmodulin spike-in experiments.

FIGS. 3A-B depict ACR (panel A) and AUR (Panel B) for cases (open squares) and controls (open circles) among Pima Indians. The dashed lines represent the 95% confidence limit relative to controls.

FIGS. 4A-C depict prognosis by combination of ACR (panel A) or AUR (Panel B) or TUR (Panel C) with HbA1C. The standard deviation for the control group was determined for each biomarker and the value summed for each of the combinations. The dashed line shows the 95% confidence limit for prognosis of future kidney disease (Bonferroni correction applied).

FIGS. 5A-B depict albumin/uromodulin ratio among groups of healthy and diabetic Caucasians from the Midwestern US. Thin females (X) and males (+) (overall ave BMI=24.6+/−1.7), obese females without diabetes (open triangle), obese males without diabetes (open squares, overall ave BMI=40.6+/−2.7), diabetic females (solid triangles) and diabetic males (solid squares) (overall ave BMI=38.7+/−3.9). The dashed line is 2 SD above the thin males and females.

FIGS. 6A-B depict AUR before and at the end of a GFR measurement. Each line represents one individual and the AUR before and at the end of the GFR test.

FIG. 7 depicts expected results for peptide quantification by MALDI-TOF mass spectrometry. The peptide of uromodulin at m/z=914.46 is shown along with the expected profile for a sample to which an identical peptide containing 5 ¹³C atoms has been added (heavy line at a monoisotopic peak of 919.5).

FIGS. 8A-C depict separation and quantification of peaks on the ESI-TOF mass spectrometer. The total ion current from the ESI-TOF is shown (upper panel) along with the elution of a peptide at m/z=301.67+/−0.02 (middle panel, extracted elution of 301.67+2 charge state of DLNIK (SEQ ID NO:2) from uromodulin) and the MS spectrum at the center of that peak (bottom panel; showing both normal peptide at 301.67 and the heavy atom peptide at 305.18). This result was typical of most peptides for albumin and at least some for uromodulin.

FIG. 9 depicts the analysis of a transferrin peptide NPDPWAK (SEQ ID NO:24) in a urine digest (trypsin) that is eluted from a reverse phase column. The parent ion at m/z=414.206 was fragmented to ions eluting at 1.84 minutes from the column at m/z=501.3, 616.3, 713.4 (top to bottom peak intensities).

FIG. 10 depicts the correlation of TUR with BMI. Solid circles are controls of the Pima Indian study, open squares are cases. The line drawn is a trendline (Excel) for cases. The dashed line is 2 SD above the controls.

FIG. 11 depicts tryptic peptide masses >500 Daltons of albumin and their charge. Peptides containing Cysteine have been modified with iodoacetamide.

FIG. 12 depicts peptide masses of >500 Daltons from uromodulin after trypsin digestion.

FIG. 13 depicts albumin and uromodulin determined by heavy atom peptide spike-in. Error bars are set at 12%, the standard deviation for replicates for several comparisons.

### DETAILED DESCRIPTION OF THE INVENTION

Based on the assays/methods provided herein, a prognosis of future kidney disease as defined by proteinuria is capable at much earlier times than currently available clinical tests. In one embodiment, the methods disclosed herein utilize protein levels relative to uromodulin (ratio) present in urine.

### Definitions

As used herein, the terms below are defined by the following meanings:

Unless defined otherwise, all technical and scientific terms used herein have the same meaning as commonly understood by one of ordinary skill in the art to which this invention belongs.

The articles “a” and “an” are used herein to refer to one or to more than one (i.e. to at least one) of the grammatical object of the article. By way of example, “an element” means one element or more than one element.

As used herein, “kidney disease” refers to a person with decreased kidney function. There are several stages of kidney disease and several definitions that are based on any of several measures including: lowered glomerular filtration rate (GFR), increased serum creatinine concentrations, elevated albumin excretion rate or elevated albumin to creatinine concentration in the urine. One definition presents 5 stages of chronic kidney disease defined by GFR with cutpoints of 90, 60, 30 and 15 mL/min/1.73 m². For purposes of this document, persons with kidney disease are defined as those with persistent albuminuria or proteinuria over 3 months (ACR>300 ug albumin (or total protein)/mg creatinine or AER>200 micrograms/min) or those with GFR<60 mL/min/1.73 m² for 3 months. Early kidney disease is defined as those with an elevated ACR over at least 3 months (>30 micrograms per mg creatinine) or elevated AER (>20 micrograms albumin per minute). The earliest stages of kidney disease precede the current definition of early stage kidney disease and are generally undetected by current methods. This stage that is undetected by current methods is also referred to as early stage kidney disease. Chronic kidney disease is defined as persons displaying the properties of kidney disease for at least 3 months.

The proteins described in this document are identified by accession numbers: human serum albumin or referred to as simply albumin (gi|4502027), uromodulin (gi|59850812), transferrin (gi|4557871), kininogen 1 or simply kininogen (gi|4504893), epidermal growth factor (gi|4503491), alpha-2-glycoprotein-Zinc (gi|4502337), orosomucoid (gi|9257232), and leucine-rich alpha-2-glycoprotein (gi|16418467).

A “subject” is a vertebrate, such as a mammal, including a human. Mammals include, but are not limited to, humans, farm animals, sport animals and pets. Included in the terms animals or pets are, but not limited to, dogs, cats, horses, rabbits, mice, rats, sheep, goats, cows and birds.

The term “biological sample,” as used herein, refers to samples obtained from a subject, including, but not limited to, skin, hair, tissue, blood, plasma, serum, cells, sweat, saliva, feces, tissue and/or urine.

The term “about,” as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. In one aspect, the term “about” means plus or minus 20% of the numerical value of the number with which it is being used. Therefore, about 50% means in the range of 45%-55%. Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The term “isolated” refers to a compound, including antibodies, nucleic acids or proteins/peptides, or cell that has been separated from at least one component which naturally accompanies it.

As used herein, “treat,” “treating” or “treatment” includes treating, reversing, ameliorating, or inhibiting an injury or disease-related condition or a symptom of an injury or disease-related condition. In one embodiment the disease, injury or disease related condition or a symptom of an injury or disease related condition is prevented; while another embodiment provides prophylactic treatment of the injury or disease related condition or a symptom of an injury or disease related condition. A “preventive” or “prophylactic” treatment is a treatment administered to a subject who does not exhibit signs, or exhibits only early signs, of a disease or disorder. A prophylactic or preventative treatment is administered for the purpose of decreasing the risk of developing pathology associated with developing the disease or disorder.

As used herein, “health care provider” includes either an individual or an institution that provides preventive, curative, promotional or rehabilitative health care services to a subject, such as a patient. In one embodiment, a health care provider is informed of the outcome of the assay.

An “effective amount” generally means an amount which provides the desired effect. For example, an effective dose is an amount sufficient to affect a beneficial or desired result, including a clinical result. The dose could be administered in one or more administrations and can include any preselected amount. The precise determination of what would be considered an effective dose may be based on factors individual to each subject, including size, age, injury or disease being treated and amount of time since the injury occurred or the disease began. One skilled in the art, particularly a physician, would be able to determine what would constitute an effective dose. Doses can vary depending on the mode of administration, e.g., local or systemic.

The terms “comprises,” “comprising,” and the like can have the meaning ascribed to them in U.S. Patent Law and can mean “includes,” “including” and the like. As used herein, “including” or “includes” or the like means including, without limitation.

### Detection/Diagnosis of Kidney Disease

Several studies have focused on better assays for early detection of kidney disease (1-7). The study of small peptides in the urine of diabetic subjects and those with chronic kidney disease (CKD) revealed elevation of several collagen peptides and a uromodulin peptide (8). Increase of peptide degradation products could arise from either elevated proteins or increased protease digestion. A recent study applied SELDI mass spectrometry to diabetic subjects and found changes in samples collected 10 years prior to diagnosis of proteinuria (9). While the latter suggested that early prognosis was possible, the method did not provide protein identification and the associated benefits such as the ability to develop methods to target specific proteins and develop alternative assays to test and corroborate the findings.

As the most abundant protein of urine, uromodulin or Tamm-Horsfall protein has been investigated with respect to its potential role in kidney disease as well as its overall function. Mice with a uromodulin gene knockout show increased susceptibility to bladder infections and kidney stone formation (26), but otherwise show little adverse effect. Recent attention has focused on human structural variants that alter uromodulin synthesis and are linked to kidney disease. Examples include a variant with lowered uromodulin production that is associated with development of chronic kidney disease (21). This finding appeared contradictory to another recent study of uromodulin variants where elevated levels of uromodulin were associated with future development of CKD and a protective gene resulted in lower excretion of uromodulin. The authors concluded that higher uromodulin excretion characterized future development of CKD (22).

Other studies have suggested that lower excretion of uromodulin increases risk for renal failure and cardiovascular disease in persons with type 1 but not type 2 diabetes (23). This appeared to contrast with reports that increased urinary concentrations of uromodulin in children were a sign of kidney dysfunction (24). Other studies suggest that elevated interstitial uromodulin is associated with inflammation and eventual decline of uromodulin excretion that predates CKD so that uromodulin may be an active player in CKD (25).

This analysis of recent reports indicates considerable uncertainty of the function of uromodulin, its concentration and/or its utility in diagnosis of disease. For example, one study reported that the uromodulin concentration in urine of control subjects gave an average of 45 mg/L with a range of 9.4 to 192.5 mg/L (Sejdiu and Torffvit, Scandinavian Journal of Urology and Nephrology 42, 168-174, 2008). Another study reported the average urine concentration of uromodulin among controls was 6.2 mg/L with quartile 1=3.5 and quartile 3=13 (Kottgen et al., J. Am. Soc Nephrol. 21, 337-344, 2010). The large range of values in both cases offered an opportunity to observe correlation between urinary albumin and uromodulin. However, no correlation between urinary albumin and uromodulin was found in either control or diabetic subjects (Torffvit et al. Clinica Chimica Acta 205, 31-41, 1992). Current knowledge therefore contradicts a key aspect of this invention. Another report suggested an average uromodulin excretion rate among healthy subjects of 63 micrograms per minute or 3.8 mg/hr (Torffvit O, Agardh C D, Thulin T. Scand J Urol Nephrol. 1999 June; 33(3):187-91) while a different report indicated an average uromodulin excretion rate of 1.3+/−0.25 mg/hr in males and 0.9+/−0.3 mg/hr in females (Nishimaki J, Masuda M, Katoh S, Nakajima T, Kanamori K, Shimomura H, Shiba K., Rinsho Byori. 2008 October; 56(10):862-7, article in Japanese).

Despite great divergence in reported concentration of uromodulin in the urine, a consistent feature of data analysis is consideration of uromodulin as an independent marker. The uromodulin concentration is expressed as excretion per unit time or is standardized to urinary creatinine in the same manner of urinary albumin. In contrast, the invention disclosed herein is based on the observation of a linkage, for example, of urinary albumin or transferrin to uromodulin. This approach indicates that uromodulin excretion can either increase or decrease in association with various conditions and that the important aspect for prognosis of kidney disease is that albumin changes in synchrony in healthy individuals. The constant AUR or TUR in healthy individuals provides a very sensitive method for detection of the earliest stage at which abnormal albumin appears in the urine. The absolute concentration of transferrin, uromodulin or albumin is secondary to the ratio of the components.

Comments regarding albumin can also be applied to transferrin in the urine. In some studies, transferrin is believed to be equivalent or slightly better than albumin in detection of kidney disease (28). It is also taken up in the proximal tubules (Renata Kozyraki, John Fyfe, Pierre J. Verroust, Christian Jacobsen, Alice Dautry-Varsat, Jakub Gburek, Thomas E. Willnow, Erik Esc) Christensen, and Soren K. Moestrup, Megalin-dependent cubilin-mediated endocytosis is a major pathway for the apical uptake of transferrin in polarized epithelia PNAS 2001 98 (22) 12491-12496). Like albumin, transferrin is currently analyzed as an independent risk factor and is standardized to its rate of excretion or to urinary creatinine.

The current study utilized the samples of the Pima Indian population that were described for SELDI analysis above (9), but applied iTRAQ technology for quantification of protein ratios. The iTRAQ method identifies and quantifies proteins in a single step, leading to immediate knowledge of both biomarkers and possible mechanisms of disease. The findings showed that protein ratios provided a sensitive approach to detect early stage kidney disease, consisting of a small decline of the kidney-specific protein, uromodulin, with a small relative increase of plasma proteins in the urine.

A second aspect of current understanding is that the appearance of albumin in urine is thought to arise from imperfect function of the kidney. That is, albumin is retained by the glomerulus and any that is filtered is taken up by specific transport in the proximal tubules. It is thought that albumin in the urine represents escape of both events. Current methods fail to detect any albumin in the urine of some individuals. The most sensitive methods for detection of urinary albumin use antibodies directed to the intact protein. An aspect of discovery that led to this invention was that antibody recognition of intact protein is challenged by the state of proteins in the urine where they can be masked from antibody recognition through minor oxidation or proteolysis or by association with another protein, such as uromodulin. In fact, an aspect of this invention is the demonstration that healthy individuals excrete albumin into their urine and that the level, expressed relative to uromodulin, is very constant among the population who have fully functional kidneys. Another aspect to this discovery was that the common practice of centrifuging urine before storage to remove cells and other solids effectively alters the amount of uromodulin due to the presence of some uromodulin in particles sufficient to be sedimented by centrifugation. While it is known that some uromodulin is lost during centrifugation, the most common practice in the field is to centrifuge nevertheless. A commercial ELISA assay for uromodulin instructs to centrifuge urine before analysis (MDBioproducts description of urine sample preparation, “General procedure (non-treated):To remove particles and debris that would interfere with analysis, filter sample through 0.45 um syringe filter or centrifuge at 2,000 g for 10 minutes and collect supernatant. Alternatively, this procedure does include methods that do not require centrifugation or filtration. “General procedure (treated to enhance solubilization): Gently mix urine sample to suspend particles/solutes that may have settled. Dilute sample between 1:25 or 1:200 (v/v) in TAE (triton, EDTA, alkaline) Buffer. TAE Buffer contains 0.5% triton x-100 and 20 mM EDTA (pH 7.5)”. At no time do the instructions indicate the best method or that a ratio of other proteins such as albumin or transferrin to uromodulin is an effective method for prognosis of future kidney disease.

The invention is based on the discovery of a constant albumin to uromodulin ratio (AUR) in healthy individuals. This consistency also applies to transferrin, and several other proteins present in urine, to uromodulin. The AUR is a very sensitive measure to detect the earliest stages of albumin increase. While BMI or other factors may alter urine concentration, pH or other properties of urine, these factors can be measured and corrected when determining the normal AUR for an individual. As a result, a small increase of the albumin or transferrin to uromodulin ratio signals a substantial change in kidney function and provides superior prognosis of future kidney disease. The invention also shows that the transferrin to uromodulin ratio (TUR) may have a slight advantage over AUR in some cases.

For example, one embodiment of the present invention provides for prognosis/diagnosis of early kidney disease, wherein the ratio of a protein (e.g., albumin or transferin) to uromodulin is greater than or equal to about 2 standard deviations above the average for a control population.

In another embodiment, the present invention provides a method for diagnosing or predicting the risk of developing kidney disease by determining the ratio of two proteins present in the urine of a subject, that the ratio is indicative of having or at risk of developing kidney disease. For example, a subject has or is at risk of developing kidney disease if the ratio for the first protein, e.g., albumin, to uromodulin is greater than about 0.30 (w/w), such as about 0.25, about 0.26, about 0.27, about 0.28, about 0.29, about 0.30, about 0.31, about 0.32, about 0.33, about 0.34, about 0.35, about 0.36, about 0.37, about 0.38, about 0.39, about 0.40, about 0.45, about 0.50, about 0.55 and higher.

It is known that urinary albumin increases in persons as they progress to kidney disease. The albumin level is commonly standardized to urinary creatinine, a small molecule that is used to normalize for the concentration of the urine. Significant levels of urinary albumin are referred to as microalbuminuria. The lower ACR limit for microalbuminuria is set at 30 micrograms of albumin per mg of creatinine or the excretion of more than 20 micrograms of albumin per minute. Most healthy individuals have lower than 30 ACR. However, variation of ACR is substantial so that a value of 30 is used in order to avoid excessive false positives. Prognosis of early stage kidney disease is often based on persistent microalbuminuria or sequential observation of ACR>30. Some events of elevated albumin are entirely reversible and do not reoccur. More accurate methods of estimating excessive albumin in the urine could enhance early diagnosis of individuals subject to future kidney disease, either from a single assay or multiple assays.

Therefore, an aspect of this invention is accurate quantification of the proteins of interest. Prior studies have shown that urinary proteins pose numerous challenges for quantification at the level of the intact protein. Freeze-thaw of urine can change the protein concentration as detected by antibodies directed to the intact proteins. As a result, one embodiment of the invention employs antibodies to intact proteins only in special cases, such as those where samples are analyzed immediately following urine collection or in samples that have been treated to eliminate aggregation and proteolysis. In some cases, the assays will target peptides that have been released by quantitative proteolysis of the proteins by protease enzymes. The assays can include mass spectrometry methods for quantification of peptides released after protease digestion of the urine proteins. Alternatively, antibodies can be developed that recognize the released peptides and can then be analyzed according to antibody assay methods. Protease digestion after disulfide reduction and alkylation removes the problems of protein aggregation and the effect of partial degradation that result in poor recognition by antibodies to the intact proteins.

In one embodiment, the detection and/or quantification of proteins is carried out by an immunoassay. An immunoassay is a biochemical test that measures the presence or concentration of a substance that frequently contain a complex mixture of substances. Analytes in biological samples, such as feces, serum or urine are frequently assayed using immunoassay methods. Such assays are based on the unique ability of an antibody to bind with high specificity to one or a very limited group of molecules. A molecule that binds to an antibody is called an antigen. Immunoassays can be carried out for either member of an antigen/antibody pair. In either case the specificity of the assay depends on the degree to which the analytical reagent is able to bind to its specific binding partner to the exclusion of all other substances that might be present in the sample to be analyzed. In addition to the need for specificity, a binding partner must be selected that has a sufficiently high affinity for the analyte to permit an accurate measurement. The affinity requirements depend on the particular assay format that is used.

In addition to binding specificity, the other feature of all immunoassays is a means to produce a measurable signal in response to a specific binding. Most immunoassays depend on the use of an analytical reagent that is associated with a detectable label. A large variety of labels have been demonstrated including radioactive elements used in radioimmunoassays; enzymes; fluorescent, phosphorescent, and chemiluminescent dyes; latex and magnetic particles; dye crystallites, gold, silver, and selenium colloidal particles; metal chelates; coenzymes; electroactive groups; oligonucleotides, stable radicals, and others. Such labels serve for detection and quantitation of binding events either after separating free and bound labeled reagents or by designing the system in such a way that a binding event effects a change in the signal produced by the label.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

Immunoassays include, but are not limited to, Enzyme-linked immunosorbent assay (ELISA), lateral flow test, latex agglutination, other forms of immunochromatography, western blot, and/or magnetic immunoassay.

Finally, it is disclosed herein that glycoproteins can provide prognosis of kidney disease, with correlation between glycoprotein to uromodulin ratios and BMI among cases, but not controls. These glycoproteins, which include but are not limited to, alpha-2-glycoprotein-Zinc, orosomucoid 1 or 2, and leucine-rich alpha-2-glycoprotein, may be used for prognosis of obese persons of lower BMI (BMI=30 to 35) who progress to kidney disease. Glycosylation levels can also be used for diagnosis of liver disease or for examination of any organ that is the origin of a given glycoprotein.

### Computer/Processor

The detection and/or diagnosis method can employ the use of a processor/computer system. For example, a general purpose computer system comprising a processor coupled to program memory storing computer program code to implement the method, to working memory, and to interfaces such as a conventional computer screen, keyboard, mouse, and printer, as well as other interfaces, such as a network interface, and software interfaces including a database interface find use one embodiment described herein.

The computer system accepts user input from a data input device, such as a keyboard, input data file, or network interface, or another system, such as the system interpreting, for example, the ELISA data or mass spectrometry data, and provides an output to an output device such as a printer, display, network interface, or data storage device. Input device, for example a network interface, receives an input comprising detection of the proteins described herein and/or quantification of those proteins. The output device provides an output such as a display, including one or more numbers and/or a graph depicting the detection and/or quantification of the proteins.

Computer system is coupled to a data store which stores data generated by the methods described herein. This data is stored for each measurement and/or each subject; optionally a plurality of sets of each of these data types is stored corresponding to each subject. One or more computers/processors may be used, for example, as a separate machine, for example, coupled to computer system over a network, or may comprise a separate or integrated program running on computer system. Whichever method is employed these systems receive data and provide data regarding detection/diagnosis in return.

### Antibodies/Detection Agents

Antibodies to detect the desired proteins can be produced by methods available to an art worker or purchased commercially.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin subunit molecules. The antibodies in the present invention may exist in a variety of forms including, for example, polyclonal antibodies, monoclonal antibodies, Fv, Fab and F(ab)₂, as well as single chain antibodies and humanized antibodies. As used herein, the term “secondary antibody” refers to an antibody that binds to the constant region of another antibody (the primary antibody).

### EXAMPLES

The following examples are provided in order to demonstrate and further illustrate certain embodiments and aspects of the present invention and are not to be construed as limiting the scope thereof.

### Example 1

### Analysis of Proteins at the Peptide Level by Mass Spectrometry Methods: iTRAQ Approach

Protein Preparation and iTRAQ Labeling.

Except where indicated, urine samples were collected and frozen without centrifugation, processing or addition of preservatives. For analysis, urine samples were thawed, mixed vigorously and a representative sample taken for analysis. Typically, 2 mL of urine was warmed to 37 degrees to solublize most precipitated material. The samples were concentrated to 0.1 mL in a spin cartridge (Millipore, cutoff of 3600 Da), microdialyzed against ammonium bicarbonate, frozen and lyophillized. Protein powders are dissolved in iTRAQ dissolution buffer and processed as described (10). Equal amounts of protein from each sample, as determined by BioRad protein assay (BioRad Laboratories, Inc.), were digested with sequence grade trypsin and each was labeled with a different iTRAQ reagent. The digested samples were mixed and the peptides fractionated by strong cation exchange. Twenty six fractions were collected. Most peptides were found in 13 fractions, every other fraction (n=7) was subjected to capillary reverse phase separation and spotted on a MALDI target by the Tempo™ spotting system. MALDI plates were submitted to a 4800 MALDI TOF/TOF™ analyzer for peptide identification and relative quantification from the marker ions at m/z=113, 114, 115, 116, 117, 118, 119, and 121 using ProteinPilot™ software (Version 2.0, ABI). A total of 1760 spots were analyzed with MS/MS analysis of the top 20 ions in each spot. The data were searched against the NCBI Human Reference Sequence database (October, 2007, 32,850 entries) using the thorough search option with fixed MMT modifications on Cys residues. Proteins identified with 67% identification confidence (>1.3 Unused Score) were used for quantification. Data were corrected by the software ‘bias factor’ that adjusts the signal for unequal amounts of protein in different samples. Typical bias factors were 0.8 to 1.2. All runs of samples from the Pima Indian study and the thin or obese adult groups (described below) were analyzed in 8-plex iTRAQ runs. Samples from non-diabetic controls were analyzed by either 8-plex or 4-plex iTRAQ reagents. The number of proteins identified at the 95% confidence limit ranged from 93 to 380. Error factor for the proteins used in this study were all well within the acceptable limit (<2.0).

In addition to individual samples, pooled samples from the Pima Indian study described below were examined. These were all females, younger cases (n=9, average BMI=37.7+/−4.4, age 37.5+/−7.0, duration of diabetes 6.7+/−2.8) and a diabetic control group (n=7, average BMI=37.4+/−6.4, age=38.8+/−3.9, and duration of diabetes=6.7+/−3.0). Another pool of cases (n=8, average age=52.0+/−3.1, BMI=34.9+/−5.0, duration of diabetes=8.9+/−3.3) and diabetic controls (n=7, average age=51.0+/−3.1, BMI 36.4+/−3.9 and duration of diabetes=7.3+/−2.9) was also analyzed. The pooled samples include eight of those who were not analyzed as individuals due to insufficient urine volume.

Standard Sample and Replicate Comparisons.

Every iTRAQ experiment contained the same standard sample prepared from urine of a healthy individual. Inclusion of this standard in every run and expression of all protein ratios relative to this standard allowed comparison of results for case and control samples that were collected in multiple iTRAQ runs. Replication of one sample ratio was estimated by inclusion of a second common urine protein sample in 12 separate iTRAQ runs. The ratio of proteins to the standard across these runs provided an estimate of reproducibility in repeated assays. In addition, earlier studies reported parallel processing, labeling and analysis of 4 duplicate samples from different individuals. The coefficient of variation for all proteins in these replicates averaged 7%. Finally, one sample from the Pima Indian study was processed on two occasions and included in two different iTRAQ experiments. Key protein ratios, expressed relative to the standard, showed values for the two analyses of: albumin, 0.47, 0.43; transferrin, 0.69, 0.74; uromodulin, 0.30, 0.28, respectively.

Software provided with iTRAQ technology was used to estimate the ratio of proteins in each iTRAQ run. This software reports the term “Error Factor (EF)”, which is the 95% confidence interval for a given protein ratio. The lower and upper limits for the 95% confidence interval are defined by equations 1 and 2. The lower limit=reported protein ratio/EF (eq. 1). The upper limit=reported protein ratio*EF (eq. 2).

A second method of data analysis compared the level of one protein to another in the same sample. This was accomplished by the relationship shown for albumin and uromodulin in equation 3. (Sample albumin/Standard albumin)/(Sample Uromodulin/Standard Uromodulin) (eq. 3). This approach effectively assigned each protein of the standard a value of 1.0.

This is the first application of internal comparison by equation 3 for iTRAQ analysis. The relative internal protein ratios identified by equation 3 are given in the text without error bars. Replicate measurements as well as intra- and inter-subject variation are presented to provide an indication of the reproducibility and variation that should be expected for ratios generated from equation 3.

Reported data were also modified by “bias factor”. This term provides a single multiplier that gives a value of 1.0 for the average of all protein ratios. Bias factor will correct for unequal amounts of protein in two samples. However, this factor assumes that the samples are nearly identical and may present some disadvantages. Application of equation 3 eliminated the impact of bias factor since the term applied equally to the numerator and denominator of equation 3. In addition, equation 3 eliminated the impact of other proteins in the urine. For example, the presence of a contaminating protein such as keratin would lower the amounts of albumin and uromodulin relative to total protein to the same extent so that equation 3 would give the same value in the presence or absence of keratins.

Statistical values from population comparisons were obtained by Student's two-tail t test. Comparison of matched pairs used paired two sample for means. Correlation coefficients were estimated from excel; p-values represent non-directional probability. In the case where two biomarkers were used to evaluate group comparison, the 95% confidence limit was established using the Bonferroni correction.

This study focused on a small portion of proteins that were quantified by iTRAQ analysis. The focus proteins (Table 1) have been implicated in kidney health. Increase of albumin is the standard marker of kidney dysfunction. Urinary transferrin has been reported to give results indistinguishable from albumin (11). The glycoproteins, alpha-2-glycoprotein-Zinc (e.g. (12, 13)), orosomucoid (14) and leucine-rich alpha-2-glycoprotein (15), have all been implicated as biomarkers of disease. Alpha-2-glycoprotein-zinc was reported to increase earlier than albumin (13).

The focus proteins are relatively abundant in urine and provided a large number of peptides that were used to determine protein ratios (Table 2). Error factor improved as the number of peptides increased (Table 2). The average number of peptides of albumin (n=133, Table 2) was 8.9-times the average for transferrin (n=15, Table 2). If quantified by peptide spectral counting, this difference in peptide number was somewhat lower than the relative abundance of albumin (approximately 40 mg/mL) and transferrin (2.5 mg/mL) in plasma.

TABLE 1
Proteins of non-diabetic controls relative to the standard sample
(samples described in Example 5).

| | A. Replicate | B. Average of Intra- | C. Average of all | D. Average |
|---|---|---|---|---|
| | (n = 12) assays | person | samples | protein/Uromodulin |
| Protein | (SD, CV %)^{a} | ratios (SD)^{b} | (SD, CV %)^{c} | (SD, CV %)^{d} |
| Albumin | 0.89 (0.11, 12) | 1.0 (0.19) | 1.11 (0.27, 24) | 1.36 (0.49, 36) |
| Transferrin | 0.98 (0.18, 18) | 1.0 (0.23) | 0.97 (0.23, 24) | 1.18 (0.45, 38) |
| Uromodulin | 0.72 (0.18, 25) | 1.0 (0.21) | 0.87 (0.23, 26) | — |
| Alpha-2-GP, | 0.87 (0.20, 23) | 1.0 (0.34) | 1.05 (0.56, 53) | 1.33 (0.94, 71) |
| Zn | | | | |
| Orosomucoid | 0.72 (0.25, 35) | 1.0 (0.36) | 2.16 (1.58, 73) | 2.48 (2.51, 101) |
| Leucine-rich | 0.74 (0.18, 24) | 1.0 (0.48) | 1.59 (1.16, 73) | 2.27 (2.26, 100) |
| alpha-2 GP | | | | |

^{a}The same sample was included in 12 different iTRAQ runs.
^{b}The average value was assigned 1.0 for each individual.
^{c}37 samples from 4 individuals collected over a 12 month period expressed as the ratio to the standard.
^{d}Ratios are expressed relative to the same proteins in the standard.

TABLE 2
Pima Indian samples (samples described in example 3). Protein
ratios are expressed relative to the standard sample.

| | Peptides for | Error | Ave. all cases | Ave. all Controls | | |
|---|---|---|---|---|---|---|
| | quantification | factor | (n = 23) | (n = 19) | Cases/ | |
| Protein | (SD) | (SD) | (SD, CV %) | (SD, CV %) | Controls | p |
| Albumin | 133 (44) | 1.19 (0.06) | 0.90 (0.33, 37) | 0.75 (0.22, 29) | 1.20 | 0.08 |
| Transferrin | 15 (5) | 1.38 (0.17) | 1.32 (0.47, 36) | 1.05 (0.29, 28) | 1.25 | 0.04 |
| Uromodulin | 66 (24) | 1.32 (0.10) | 0.46 (0.15, 33) | 0.60 (0.14, 23) | 0.78 | 0.006 |
| Alpha-2-GP, | 23 (5) | 1.38 (0.16) | 1.58 (0.89, 56) | 1.36 (0.89, 65) | 1.16 | 0.43 |
| Zn | | | | | | |
| Orosomucoid | 15 (5) | 1.66 (0.38) | 1.13 (0.63, 56) | 1.03 (0.58, 56) | 1.10 | 0.58 |
| 1 | | | | | | |
| Leucine-rich | 4.8 (2.1) | 1.73 (0.51) | 1.40 (0.70, 50) | 1.04 (0.27, 26) | 1.34 | 0.047 |
| Alpha-2-GP | | | | | | |
| Protein | — | — | 25.7 (20) | 28.9 (32.4) | 0.89 | 0.74 |
| isolated | | | | | | |
| ug/mL urine | | | | | | |

### Example 2

### Spike-in Experiments to Determine the Absolute Concentration of Albumin and Uromodulin in the Standard Sample

Spike-in experiments were conducted with purified human serum albumin and uromodulin. Albumin was quantified by BioRad protein assay. Uromodulin was quantified by absorbance at 280 nm based on the theoretical extinction coefficient of uromodulin (1.19 Absorbance units in a 1 cm path length at 1.0 mg/mL with 7 glycosylation events, determined by EXPASY software).

One discovery allowed comparison of protein ratios within each sample. Individual protein ratios to the standard were divided by the uromodulin ratio to the standard. The iTRAQ method effectively assigned the protein concentration of the standard, the denominator in all cases, a value of 1.0 regardless of its absolute concentration. In this way, the relative ratio of albumin to uromodulin in sample A could be compared to the relative ratio in sample B by (Table 1D).

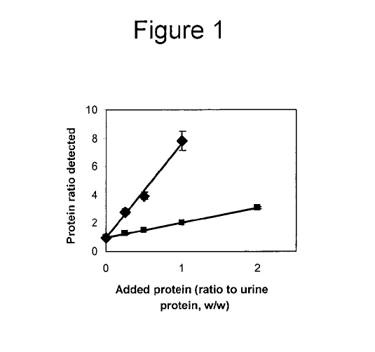

Relative protein ratios can be converted to absolute concentration in those cases where the concentration in the standard was determined by separate experiment. For example, albumin and uromodulin were independently spiked into the standard sample and the ratio of albumin to uromodulin determined. Both proteins showed linear increase of ratio with the amount of spiked protein (FIG. 1). Linear increase was expected in the case that protein ratios remained within the dynamic range of the assay and were not influenced by factors such as background intensity. The slopes of the plots, together with the amount of protein added, were used to calculate the concentration of the individual protein in the standard. From this information, albumin comprised 15.0% of the BioRad-quantified protein in the standard sample. The value obtained for uromodulin was 95%. A sum greater than 100% for all proteins was possible from the fact that uromodulin gave poor color yield in the BioRad assay (18% of the response of serum albumin). Consequently, the actual amount of protein used for iTRAQ labeling was greater than the 40 micrograms detected by the BioRad assay. Assuming that all other urinary proteins gave color yields similar to albumin, these results indicated that uromodulin comprised 48% of the total urinary protein, very nearly the commonly accepted portion of uromodulin in the urine. In any event, the ratio of albumin to uromodulin (w/w) in the standard was 0.156.

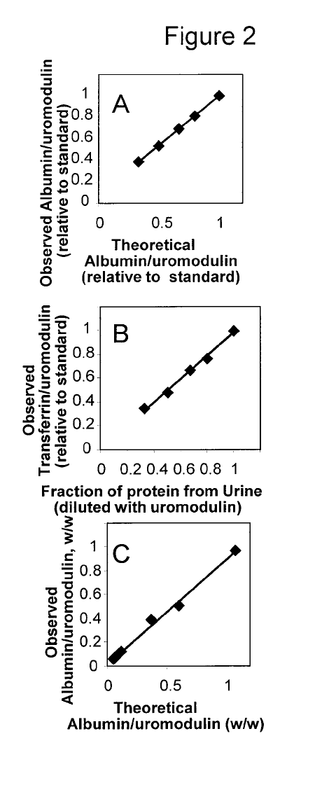

From these experiments, it was possible to express the ratio of albumin/uromodulin relative to the ratio of the standard (FIG. 2A) or as an absolute concentration (w/w, FIG. 2C). Expression as absolute concentration would not impact the observed fold-changes between groups or the statistical significance of group comparisons. Relative values were used in subsequent analyses. This allowed similar comparison of proteins for which spike-in experiments were not conducted.

The accurate dynamic range of iTRAQ analysis for this internal comparison of urinary proteins was clearly greater than 8.0 (FIG. 1). In other studies, ratios of up to 35 for albumin/uromodulin have been detected in advanced kidney disease (data not shown). The range found in the current study was within the accurate dynamic range defined in FIG. 1.

### Example 3

### Prognosis of Kidney Disease Among Pima Indians Using ACR Vs. AUR

Diabetic cases and Controls. The sample base from the Pima Indian study used in this study was described in detail elsewhere (9). Cases or progressors were persons who developed proteinuria within 10 years while diabetic controls maintained normal urinary protein throughout. In the case of matched pairs, diabetic controls were matched to cases with respect to age (+/−3 years), gender, BMI (+/−2 kg/m2) and duration of diabetes (+/−2 years). A total of 42 were analyzed by iTRAQ proteomics method, 23 progressors (cases) and 19 non-progressors (diabetic controls). The basis for sample selection for individual iTRAQ analysis was the availability of sufficient urine volume for protein analysis. Another 8 samples were analyzed by iTRAQ as part of pooled samples. Results for the pooled samples were consistent with the findings reported for individuals. Individual comparisons included 15 matched pairs. Samples had been stored frozen at −80 degrees C. until thawed for analysis. At least one freeze-thaw cycle had been applied before application of iTRAQ analysis.

Table 3 shows baseline and 10-year follow-up characteristics of cases and diabetic controls. At baseline, a significant difference in HbA1C as well as ACR was observed. At the 10-year follow-up, the difference in HbA1C had declined and the major change consisted of proteinuria among cases. At follow-up, there was a small difference in systolic blood pressure while diastolic blood pressure showed a trend (p=0.06).

TABLE 3
Characteristics at sampling time and at 10 years.

| Pima Subjects | Cases (n = 23) | Controls (n = 19) | p |
|---|---|---|---|
| Baseline Characteristics | | | |
| Age (years) | 42.0 ± 9.4 | 43.2 ± 9.0 | 0.78 |
| Sex (% Female) | 78 | 79 | |
| Systolic Blood Pressure | 122 ± 16 | 121 ± 19 | 0.93 |
| (mmHg) | | | |
| Diastolic Blood Pressure | 77 ± 10 | 75 ± 12 | 0.58 |
| (mmHg) | | | |
| Serum Creatinine (mg/dl) | 0.68 ± 0.14 | 0.73 ± 0.13 | 0.23 |
| Hemoglobin A1C (%) | 8.29 ± 2.39 | 6.33 ± 1.57 | 0.0038 |
| Urine Albumin Creatinine | 15.1 ± 8.9 | 9.6 ± 6.3 | 0.029 |
| Ratio (mg/g) | | | |
| Follow Up Characteristics | | | |
| Age (years) | 52.3 ± 9.3 | 52.8 ± 9.3 | 0.84 |
| Systolic Blood Pressure | 136.8 ± 16.2 | 120.8 ± 19.5 | 0.006 |
| (mmHg) | | | |
| Diastolic Blood Pressure | 79.2 ± 10.1 | 73.5 ± 9.0 | 0.06 |
| (mmHg) | | | |
| Serum Creatinine (mg/dl) | 0.77 ± 0.26 | 0.74 ± 0.30 | 0.62 |
| Hemoglobin A₁C (%) | 10.6+/−2.1 | 9.3+/2.0 | 0.06 |
| Urine Albumin Creatinine | 1163 ± 1097 | 16 ± 8 | 5.0E−5 |
| Ratio | | | |

Actual protein ratios relative to the control differed only slightly for cases vs. controls (Table 4) with slightly elevated albumin and transferrin and decreased uromodulin.

TABLE 4
Pima Indian samples. Protein ratios relative to the standard sample.

| | Peptides for | Error | Ave. all cases | Ave. all Controls | | |
|---|---|---|---|---|---|---|
| | quantification | factor | (n = 23) | (n = 19) | Cases/ | |
| Protein | (SD) | (SD) | (SD, CV %) | (SD, CV %) | Controls | p |
| Albumin | 133 (44) | 1.19 (0.06) | 0.90 (0.33, 37) | 0.75 (0.22, 29) | 1.20 | 0.08 |
| Transferrin | 15 (5) | 1.38 (0.17) | 1.32 (0.47, 36) | 1.05 (0.29, 28) | 1.25 | 0.04 |
| Uromodulin | 66 (24) | 1.32 (0.10) | 0.46 (0.15, 33) | 0.60 (0.14, 23) | 0.78 | 0.006 |
| Alpha-2-GP, | 23 (5) | 1.38 (0.16) | 1.58 (0.89, 56) | 1.36 (0.89, 65) | 1.16 | 0.43 |
| Zn | | | | | | |
| Orosomucoid | 15 (5) | 1.66 (0.38) | 1.13 (0.63, 56) | 1.03 (0.58, 56) | 1.10 | 0.58 |
| 1 | | | | | | |
| Leucine-rich | 4.8 (2.1) | 1.73 (0.51) | 1.40 (0.70, 50) | 1.04 (0.27, 26) | 1.34 | 0.047 |
| Alpha-2-GP | | | | | | |
| Protein | — | — | 25.7 (20) | 28.9 (32.4) | 0.89 | 0.74 |
| isolated | | | | | | |
| ug/mL urine | | | | | | |

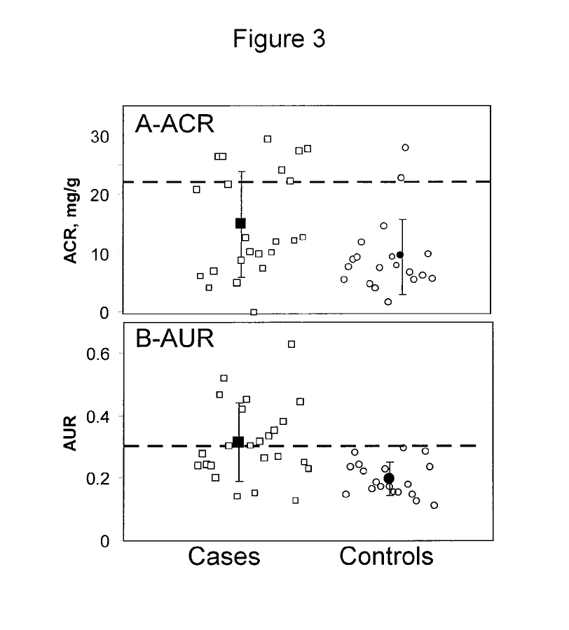

The enhanced ability for prognosis of future kidney disease among a population of Pima Indians by methods of this invention is illustrated by comparison of current methods (albumin to creatinine ratio, ACR, FIG. 3A) with the albumin to uromodulin ratio (AUR, FIG. 3B). Cases refer to Pima Indians with normal albuminuria at baseline, but who progress to kidney disease within 10 years. Controls were matched with cases with respect to age, gender, BMI and duration of diabetes but were individuals who maintained normal ACR at 10 years. The results in FIG. 3A are consistent with the current understanding that some individuals excrete almost no albumin in the urine and that the amount excreted is highly variable, even when corrected for urine concentration by reference to creatinine in ACR. The large standard deviation (CV=65%) for ACR among controls resulted in only 7 of 23 cases who were outside 2 standard deviations (SD) of the control population (the 95% confidence level) and one that was outside of 3 SD (99% confidence level). However, two of the controls were also outside the 95% limit, leading to 30% sensitivity for prognosis and 89% specificity. The defined limit for abnormal urinary albumin, defined as microalbuminuria, is 30 mg albumin/gram of creatinine. Based on current methods, all of the cases were within the accepted normal level of albumin excretion, although some were clearly elevated relative to controls. The results for AUR, TUR and other protein to uromodulin ratios are summarized in Table 5.

TABLE 5
Protein ratio to uromodulin in the same sample
(relative to the same proteins in the standard).

| Protein ratio to | | | | |
|---|---|---|---|---|
| uromodulin | Cases (SD) | Controls (SD) | Cases/controls | p |
| Albumin | 2.02 (0.80) | 1.27 (0.35) | 1.59 | 4.6*10⁻⁴ |
| Transferrin | 2.97 (1.06) | 1.81 (0.45) | 1.63 | 6.8*10⁻⁵ |
| Alpha-2-GP, Zn | 3.68 (2.28) | 2.49 (1.94) | 1.47 | 0.08 |
| Orosomucoid 1 | 2.56 (1.38) | 1.84 (1.30) | 1.39 | 0.09 |
| Leucine-Rich | 3.20 (1.80) | 1.85 (0.71) | 1.73 | 0.005 |
| Alpha-2-GP | | | | |

The ACR for cases was 1.57-times the value for controls (FIG. 3A). The AUR for cases was 1.59-times that of controls (FIG. 3B). Thus, the benefit of AUR was a more precise measure and improved internal standard for expression of albumin excretion. The results show that excretion of a limited amount of albumin is a characteristic of healthy individuals and is closely associated with uromodulin in the urine. In contrast, creatinine is excreted by an independent mechanism that results in greater variation relative to albumin. Other studies indicate a larger CV for ACR than that of the Pima Indian controls. A recent study provided a 4-fold range for ACR for control samples at the 25 and 75 percentiles (22), correlating with a CV substantially larger than that for the Pima Indian controls.

The assay provided correct prognosis at the 95% confidence limit for 12 of 23 cases and correct prognosis at the 99% confidence for 8 of 23 cases (FIG. 3B). Those with elevated AUR included 6 of the 7 with elevated ACR. The reverse was not true. Of the 5 with elevated AUR but not ACR, the average ACR was 12.5+/−4.8 mg/g, only slightly higher than controls. Ability to provide accurate prognosis for over half of cases with accurate prognosis of 74% of individuals at risk of kidney disease at 10 years prior to detection by current methods provides a powerful tool for determination of those to whom intense intervention should be applied. It is believed that prognosis will be even more accurate at 5 years prior to diagnosis by current methods.

The description above for diagnosis by ACR, AUR and TUR utilized a single assay of each individual. Current practice with ACR is the use of persistent microalbuminuria, the appearance of microalbuminuria (ACR>30 micrograms per milligram of creatinine) on at least two sequential occasions. This method of diagnosis can also be applied to AUR or TUR. That is, an elevated AUR or TUR on at least two occasions within a period of 3 months or longer. The advantages of AUR and TUR are apparent. The higher level of albumin required by the ACR will produce some individuals with elevated albumin on one occasion but who appear normal on the next. The more sensitive AUR and TUR will identify those with abnormal values with greater precision so that persistent elevation can be diagnosed with greater certainty. Overall, when used for the purpose of persistent elevation of albumin excretion, the AUR will detect persistent elevation with greater consistency and sensitivity than ACR.

Uromodulin also declines in association with coronary heart disease (Yuyun et al., American Journal of epidemiology 159, 284-293, 2004). Thus, the albumin to uromodulin or transferrin to uromodulin ratios can be useful in diagnoses/prognoses of coronary artery or cardiovascular diseases.

The Pima Indian sample group contained 15 pairs of subjects that were matched for BMI, gender, and duration of diabetes as outlined above. These 30 individuals were evaluated by two methods. One was for a difference of the mean as matched pairs (ttest: paired sample for means) and the other method as two groups without matching of individuals. The two methods of data analysis gave similar significance (Table 6). Thus, the method of analysis appeared independent of the parameters used for pair matching in the Pima Indian study. As shown below, some impact of BMI was evident at extreme differences.

TABLE 6
Analysis as matched pairs vs. group comparison.

| | | | | P for |
|---|---|---|---|---|
| | | | P for | group |
| Protein/ | Cases | Controls | pair-wise | compar- |
| uromodulin | (n = 15) | (n = 15) | compar- | ison |
| ratio | (Variance) | (Variance) | ison | (ttest) |
| Albumin | 1.90 (0.52) | 1.32 (0.14) | 0.0025 | 0.0087 |
| Transferrin | 2.85 (1.12) | 1.89 (0.19) | 0.0033 | 0.0029 |
| Alpha-2-GP, Zn | 3.53 (7.3) | 2.64 (4.7) | 0.21 | 0.32 |
| Orosomucoid 1 | 2.64 (2.64) | 1.97 (2.06) | 0.15 | 0.24 |
| Leucine-Rich Alpha- | 3.10 (4.27) | 1.69 (0.12) | 0.024 | 0.012 |
| 2-GP | | | | |
| Uromodulin alone | 0.52 (0.024) | 0.62 (0.019) | 0.015 | 0.075 |

### Example 4

### Combination of AUR or TUR with Other Measures of Risk for Developing Kidney Disease

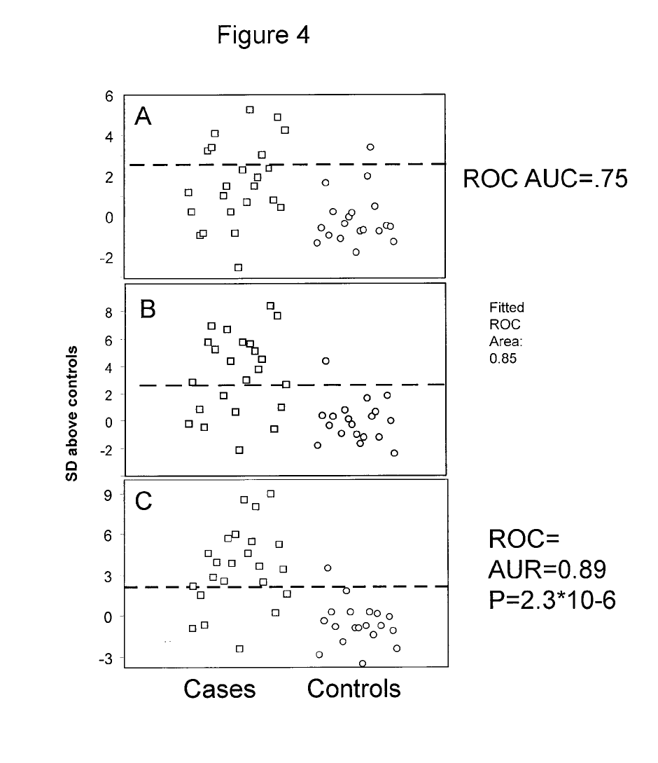

Several current measurements have some utility as risk factors for prognosis of kidney disease. These include fasting glucose level, high blood glucose during a glucose tolerance test, elevated hemoglobin A1C and elevated blood pressure. It is possible to combine these measures in various ways to generate an overall prognosis score for risk of developing kidney disease. Examples of combinations are presented for illustration purposes. These calculations utilized the Standard Deviations (SD) of controls for each biomarker. The SDs for the target measurements were summed for each sample. The results are presented in FIG. 4. The 95% confidence limit for prognosis of future kidney disease was estimated using the Bonferroni correction.

FIG. 4A shows prognosis on the basis of ACR plus Hemoglobin A1C (HbA1C) levels at baseline. Seven of 23 cases were diagnosed at the 95% confidence level (p=0.0013). FIG. 4B shows the combination of AUR with HbA1C with accurate prognosis for 15 of 23 at the 95% confidence level (p=3.7*10⁻⁵). Another combination that can be used is transferrin to uromodulin ratio (TUR) plus HbA1C at baseline. This combination resulted in accurate prognosis of 17 of 23 cases at the 95% confidence level (FIG. 4C, p=2.3*10⁻⁶). Prognosis on the basis of multiple measures may be improved further by addition of blood pressure or other estimates of kidney risk, such as fasting glucose or glucose level following the glucose tolerance test. Numerous other methods can be used to combine various markers to improve the outcomes illustrated in FIG. 4. The method of FIG. 4 assigned equal weight to biomarkers. However, some markers such as AUR and TUR had higher statistical significance and outcomes may be improved by applying higher weight to these values. In any event, the result of FIG. 4 illustrated that various combinations of risk factors can be used to improve overall prognosis of future kidney disease.

Values for cases and controls can also be evaluated by the well-known Receiver Operator Characteristic curve (ROC). The area under the curve was 0.75 for data in FIG. 4A, 0.85 for results in FIG. 4B and 0.89 for results in FIG. 4C.

### Example 5

### AUR of Other Ethnic Groups, with and without Risk of Kidney Disease

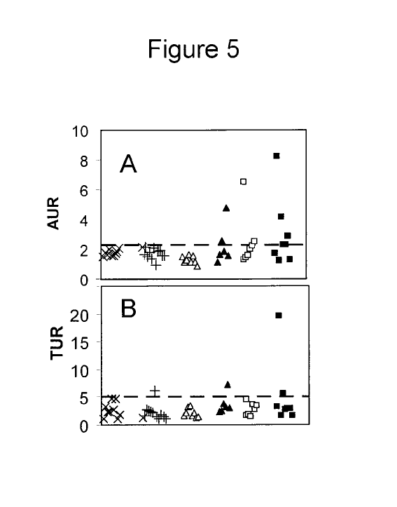

To show the general applicability of AUR and TUR for individuals of different ethnic groups, BMI and health status, a number of other subject groups were evaluated. Thin, healthy individuals were selected randomly from a larger group of individuals of the Midwestern US. These included 10 males and 10 females of average BMI=24.6+/−1.7 and average age approximately 45 years. The AUR was independent of sex (FIG. 5A) and the overall coefficient of variation was 17%, only slightly larger than the standard deviation for replicate assays by the method used (12 comparisons of the same 2 samples run in 12 different iTRAQ runs gave a sample to standard ratio of 0.88+/−0.11 or a CV of 12%). This narrow range and independence of gender indicated a very constant AUR for all healthy subjects.

Non-diabetic, obese females with normal blood fasting glucose, HDL and LDL cholesterol and triglyceride levels gave very constant values for AUR (1.29+/−0.26, CV=21%, FIG. 5A) that were indistinguishable from the controls of the Pima Indian study (1.27+/−0.35, CV=28%) (FIG. 3B). This indicated that control values were independent of ethnic group. For males with blood glucose <110 mg/dL and HDL cholesterol within the normal range, 2 of 6 gave elevated AUR values. Among obese, diabetic subjects, AUR was elevated more than 2 SD above the thin controls in 2 of 6 females and 5 of 8 males (FIG. 5A). While long-term follow-up analysis of these individuals was not available, the results were consistent with the concept that healthy controls are very constant and that a portion of those with diabetes who are therefore known to be at risk of kidney disease have elevated AUR. Those with elevated AUR will have greater probability of developing kidney disease.

TUR values for the same individuals were also determined and are illustrated in FIG. 5B. Again, controls were very consistent and a portion of those individuals known to be at risk of future kidney disease showed elevated values.

Another set of controls were studied in greater detail. Five healthy, non-diabetic volunteers each provided multiple urine samples. BMI for this group ranged from 22 to 29. These samples were collected from 2006-2009 at the University of Minnesota. A partial description of these samples has been presented (10). While detailed medical histories were not obtained, these individuals had no overt evidence of disease and were not diabetic. These controls included females (n=2) and males (n=3) ages 24 to 65 with ethnic groups comprised of Caucasian, Asian, and African American. Four of the five individuals provided 9 samples each that were collected over a 52 week period. These included one female and three males, ages 26-65 with all three ethnic groups included. The samples were collected at zero, one, two, three, 26 and 52 weeks. Four samples were obtained at the 52 week time point: first morning and afternoon urines on consecutive days. All other samples were daytime, either AM or PM, without specification. One subject provided an additional two samples at 180 and 181 weeks, the first on an occasion of unusually high urine concentration and the other at dilute concentration. In these latter samples, the amount of protein isolated was 33 and 12 micrograms per mL of original urine, respectively. These non-diabetic control samples were analyzed by intra-individual comparisons (i.e. all samples collected from each individual) over time. The average CV for albumin relative to the standard for all samples from each individual was 19% (Table 1B), slightly less than the CV for all samples relative to the standard ((24%, Table 1C).

The average and standard deviation for AUR for all samples of this multi-ethnic group was 1.36+/−0.49 (Table 1D, CV=36%), very similar to the diabetic controls from Pima and controls for obese individuals without diabetes (FIGS. 3B and 5A). Overall, the consistency of AUR among control subjects was most striking and allowed detection of very small increases that characterized those who progress to kidney disease. TUR gave similar consistency for intra- and inter-person comparison among this group (Table 1).

Overall, the consistency among controls of all types, gender and ethnic groups allows accurate detection of the earliest rise in albumin excretion and the earliest prognosis of future kidney disease.

The consistency is so significant that it appears that albumin found in the urine is linked to uromodulin excretion. While many ideas for a connection may be possible, a suggested mechanism that might link albumin with uromodulin is a protein-protein association in the proximal tubules. Complexed albumin or transferrin may be unavailable for re-uptake. In this case, escape of free albumin or transferrin into the urine may be approximately zero in healthy persons. The albumin found in urine of healthy subjects arises from this suggested protein-protein association. This basis would explain the lack of impact of age, sex or ethnic origin on AUR or TUR. Concentration of urine and pH would influence this equilibrium binding and should be considered when selecting the proper standard or control group.

### Example 6

### Use of Proper Control Groups

The non-diabetic obese females in FIG. 5 showed a significantly lower AUR (1.29+/−0.26) than the thin subjects (1.72+/−0.29, p=0.001). While these differences were small, they correlated with differences in urine concentration. The amount of protein isolated from the thin subjects was higher than that from the obese females (37.5+/−23.0 vs. 15.0+/−8.5 micrograms per mL of urine, p=0.012). Thus, it appeared possible that more concentrated urine gave higher AUR values. Thus, for optimum prognosis, subjects at risk for kidney disease should be compared to appropriately matched controls. Proper matching should include BMI, at least in the extremes of very obese (BMI>30) vs. thin subjects (BMI<25).

Other considerations for identification of the control sample group is the time of day for sample collection. Often, current procedures for urinalysis attempt to standardize urine collection. Many protocols specify first morning urine or timed-collection of urine. These requirements can be challenging for both logistical and compliance purposes. The present study indicated that spot daytime urines provided sufficient consistency, but should not be compared with first morning urines. To illustrate this aspect of sample collection, AUR was determined for first morning and afternoon urines. The samples used for comparison were collected on the same day, from the same individual (4 individuals, 2 AM/PM samples each). The first and second AM/PM urine samples were collected on consecutive days. This further minimized time-dependent change that might occur in each individual. The average ratio of AUR for first morning urine to afternoon urine of the same day was 1.09+/−0.39. The average difference was small and the CV was only 35%. First morning urines are generally more concentrated, as expected. The slightly higher AUR of morning urines may arise from the concentration effect suggested for the thin vs. obese individuals described above.

A surprising finding was that the AM/PM ratios for individuals showed greater variation than longitudinal analysis of daytime urines from the same persons over a 12 month period. The CV for intra-person AUR was determined for 7 daytime urine samples from each of the same 4 individuals described for the AM/PM studies. These daytime samples were gathered over a 12 month period. The CV values for the four individuals ranged from 19-25% (vs. 35% for AM/PM comparison). This showed that samples gathered at distant times, but as random daytime samples had less variation of AUR than samples taken as first morning vs. afternoon on the same day. Overall, optimum analysis will be obtained by use of the same sampling method for all individuals and by matching controls to cases. Current information suggests that a factor used to match cases with controls is BMI. Similar results were obtained for TUR. Random daytime urines appeared adequate to provide consistent AUR and TUR values.

One example of extreme urine concentration changes in a control individual was carried out. One sample was obtained at a very high urine concentration (33 micrograms of isolated protein per mL) and the other at unusually low urine concentration (12 micrograms of protein isolated per mL of urine). The AUR for these two samples, relative to the standard were 1.15 and 0.93. This experiment also suggested that AUR and TUR increased at higher protein concentration. This trend would be expected if protein-protein association between albumin or transferrin and uromodulin were the basis for protein appearance in the urine. More concentrated urine would result in a higher ratio of albumin- or transferrin-uromodulin complex to free uromodulin in the urine.

### Example 7

### Linkage of Urinary Proteins

Linked excretion of albumin and uromodulin or other proteins in the urine was demonstrated by several approaches. The thin adult sample base served as one example. The first approach to demonstrate linkage involved comparison of two methods for determining the overall average and SD for the protein/uromodulin ratio. Method 1 consisted of determining each individual albumin to uromodulin ratio and then calculating the average and standard deviation of the result (1.72+/−0.29, CV=17%). The other method consisted of determining the average and SD for each individual protein ratio relative to the standard and then calculating the expected average and SD for the protein/uromodulin ratio by the method for adding standard deviations. The average albumin/standard was 0.95+/−0.28 and uromodulin/standard was 0.55+/−0.10. The calculated SD for albumin/uromodulin was obtained from the relationship (Ratio of the two averages)((SD₁/average₁)²+(SD₂/Average₂)²)^{0.5}. The calculated average and SD=1.73+/−0.60 (CV=35%). The calculated SD was larger than the actual SD. The calculated SD assumes that the two values are independent of each other. The fact that the observed SD of the ratio was less than half of the calculated SD indicated that the two proteins were linked.

Statistical significance of the difference in computing SD was estimated by use of another approach. In this case, comparison of observed vs. theoretical values for the deviation of protein ratios (delta) from the mean were used. The observed delta (relative to the mean) is defined as the observed value for a protein ratio in a particular sample (X) minus the observed mean for all samples in the group under study, divided by the mean (Observed Delta=Absolute((X−Xₐᵥₑ)/Xₐᵥₑ). The theoretical Delta for each protein ratio is the combination of the Delta for protein 1 (X₁) with respect to its mean (X₁ₐᵥₑ) and the Delta for protein 2 (X₂) with respect to its mean (X₂ₐᵥₑ) by the relationship: Theoretical Delta=(((X₁−X₁ₐᵥₑ)/(Xₗₐᵥₑ))²+((X₂−X₂ₐᵥₑ)/(X₂ₐᵥₑ))²)^{0.5}. The average and SD for observed and theoretical Delta values for several of the sample groups (described above) are presented in Table 7A. Unlinked proteins are those that appear in the urine by independent mechanisms. These will be identified by indistinguishable means for observed and theoretical Delta values. For the 20 thin adult controls (described above), no linkage was detected for uromodulin with kininogen, transferrin, alpha-1-microglobulin, Zn-Alpha-2-glycoprotein, leucine-rich alpha-2-glycoprotein or epidermal growth factor (Table 7A). In contrast, the observed average Delta for the albumin/uromodulin ratio was less than half of the theoretical value (p=0.00075, Table 7A). That the ratio of albumin to uromodulin showed less than the expected Delta was an indication of linked excretion. The appearance of these proteins in the urine is dependent on one another.

This linkage was based on the same amount of protein used for each comparison and was therefore a value that was independent of the concentration of the urine. Linkage is expected between almost any urine components when both are expressed relative to urine volume. More concentrated urine will have higher concentration of virtually all components.

This linkage was found by analysis of each of the 4 control groups described above (Pima controls, thin adults, obese adults, and longitudinal analysis of 4 healthy individuals). In the overall analysis (Table 7B), some linkage was detected between uromodulin and kininogen and between uromodulin and epidermal growth factor. These three proteins originate in the kidney and some linkage may be expected. Minor linkage was detected for transferrin and uromodulin as well. It is possible that re-uptake of transferrin in the proximal tubules is less complete than albumin, leading to lower linkage between transferrin and uromodulin than was observed for albumin and uromodulin. The observed average Delta for leucine-rich alpha-2-glycoprotein was greater than the theoretical value, suggesting a negative linkage (Table 7B). In all cases, the most striking linkage was between albumin and uromodulin (p=9.1*10⁻⁷).

All possible protein ratios of Table 7A were examined in the thin adult control group (Details not shown). A weak correlation between albumin and kininogen was detected (p=0.02). In some cases kininogen may be used instead of uromodulin as the kidney standard protein. No other ratio among the proteins of Table 7A showed significant linkage. An important ratio was albumin to transferrin. As shown below, these proteins in urine were highly correlated in persons with excess albumin but were not correlated in control groups such as those in Table 7A.

Detectable linkage of albumin and uromodulin was found at somewhat elevated AUR. Twenty samples from kidney transplant recipients with no apparent complications gave an average AUR of 3.67+/−2.2 (expressed relative to the standard sample; the corresponding w/w AUR was 0.57+/−0.34). The observed Delta for this group, standardized to the average, was 0.38+/−0.47, significantly less than the theoretical average of 0.54+/−0.29 (p=0.0021). No linkage was detected for groups of samples with higher AUR averages. For example, two other groups of kidney transplant recipients were studied. One group of 33 subjects had shown adverse response and was undergoing tests for possible rejection or other complications. These had an average AUR of 11.3+/−7.9 (relative to the standard sample). The observed average theoretical Delta value (relative to the mean) was 0.50+/−0.32, virtually identical to the observed value of 0.55+/−0.51 (p=0.67). Another group of 21 transplant recipients had an average AUR of 7.4+/−5.0 (relative to the standard sample). The observed average Delta was 0.54+/−0.41 vs. a theoretical Delta of 0.63+/−0.39. Thus, the albumin-uromodulin linkage was detectable up to AUR values of about 3.0 (relative to the standard sample, w/w=0.46). It is expected that at least some of the albumin molecules are still linked to uromodulin excretion while other molecules have low or no linkage. As unlinked molecules increase, overall linkage becomes insignificant. Transplant recipients have only one kidney so that greater AUR values may still characterize a healthy organ. However, an AUR above 4 (w/w=0.62) among transplant recipients indicated significant deterioration of the transplanted organ.

A third method to illustrate linkage of proteins in the urine used correlation coefficients. In this case, the ratio of one protein to the standard was plotted versus the ratio of another protein to the standard in Excel software program and the correlation coefficient was determined. For the 20 thin healthy adults, a plot of albumin/standard vs. uromodulin/standard gave a slope of 2.17, intercept of −0.24 and R=0.81 (p<0.0001). This correlation was also significant for the eight obese female adults (p=0.035) and for the nineteen Pima Indian controls (p=0.048). Both of the latter groups gave a slope of 0.75. Plots for other proteins among the thin adult group showed significant but lower correlation of uromodulin with both epidermal growth factor and Kininogen 1. These were expected since all are at least partially of kidney origin. This approach detected a significant correlation for uromodulin with transferrin (p=0.04) but only among the Pima Indian controls. No correlation was found for uromodulin with orosomucoid or alpha-2-glycoprotein, Zn. Overall, the most significant correlation in all control groups was between albumin and uromodulin.

The findings of linkage between albumin and uromodulin are in direct contradiction to current knowledge. For example, Torffvit et al. studied albumin and uromodulin among other urinary constituents and analyzed the results by the same method. They reported that albumin and uromodulin showed no correlation in either healthy control or diabetic subject groups (Torffvit O, Agardh C D, Kjellsson B, Wieslander J., Tubular secretion of Tamm-Horsfall protein in type 1 (insulin-dependent) diabetes mellitus using a simplified enzyme linked immunoassay. Clin Chim Acta. 1992 Jan. 31; 205(1-2):31-41.)

Difference of this invention from current best practices can also be illustrated by centrifugation of the urine samples. This invention emphasizes that centrifugation of the urine sample should be avoided since it results in loss of some uromodulin in the pellet. An example was a different set of 13 samples from obese individuals prior to or after bariatric surgery. These individuals had normal kidney function as determined by serum creatinine levels. However, the samples had been centrifuged before freezing. The average AUR was 4.98+/−2.2 (relative to the standard sample), the observed Delta for AUR was 0.33+/−0.28 and the theoretical Delta was 0.45+/−0.33 (p=0.35). Although uncentrifuged control samples were not available, the average AUR exceeded the level for the healthy kidney transplant recipients (3.67, above) where linkage was still detectable. In another case of samples from transplant recipients for whom samples were centrifuged before analysis, 16 persons with normal protocol biopsies have an average AUR of 6.95+/−5.8. The observed Delta was 0.58+/−0.34 versus the theoretical Delta of 0.73+/−0.39 (p=0.26). The difference between this group and the earlier group of transplant recipients (average Delta=3.67, above) illustrated the adverse impact of centrifugation on measurement of AUR.

That the importance of the AUR is not generally appreciated is indicated by many common protocols for urine collection in major studies, where centrifugation before freezing is a common practice. For example, 6 of 7 studies that were examined centrifuged the urine before freezing. These included “Diabetes Control and Complications Trial”, “Epidemiology of Diabetes Interventions and Complications, CINCY (Cincinnati study), MSSM (Mount Sinai School of Medicine) Donor, Ohio SLE (Systematic Lupus Erythematosus) Study, and CRIC (Chronic Renal Insufficiency Cohort). A study that did not centrifuge was AASK (African American Study of Kidney Disease). Furthermore, a recent study of biomarkers for Kidney transplant rejection between the University of Minnesota and Mayo Clinic (2006-2008, Drs. Cosio and Oetting, Principal investigators) centrifuged the urine samples before freezing. Centrifugation of urine before storage is considered to be a best practice and uncentrifuged samples may be viewed as having lower stringency. While centrifugation appears useful for removal of cells that may rupture upon freeze-thaw, it has a well-established adverse impact on uromodulin concentration and therefore the AUR ratio.

Many studies have shown that centrifugation of urine results in significant loss of uromodulin (e.g. K. Kobayashi and S. Fukuoka, Conditions for Solubilization of Tamm-Horsfall Protein/Uromodulin in Human Urine and Establishment of a Sensitive and Accurate Enzyme-Linked Immunosorbent Assay (ELISA) Method, Archives of Biochemistry and Biophysics, Volume 388, Issue 1, 1 Apr. 2001, Pages 113-120.). That best practices virtually always call for urine centrifugation is illustrated by two recent documents from the National Institutes of Diabetes and Digestive and Kidney Diseases (NIDDK). One presentation calls for immediate urine processing by centrifugation for 10 minutes (Slide 2 of the presentation by Dr. Sushrut Waikar at the Third Meeting of the NIDDK-sponsored Chronic Kidney Disease Biomarkers Consortium, Apr. 2, 2010, web site reference http://sites.google.com/site/ckdbiomarkerscontortium/file-cabinet, the file titled stability presentation_final.ppt). The recommendations for ideal practice consisted of the following steps: immediately process; centrifuge×10 min; aliquot; freeze at −80° C.; always at −80° C.; no freeze-thaw cycle; and assay within reasonable timeframe.

A second, authoritative source contains a summary of recommendations arising from a meeting specifically devoted to urine collection and storage entitled “Best Practices for Sample storage: Urine as a Paradigm. Workshop on Urine Biospecimen Handling” sponsored by the NIDDK on Feb. 22-23, 2010 in Bethesda Md. This included leading experts on kidney diseases and sample handling methods. The report of working group 2 was entitled: “Collection, Handling and Long-term storage of urine” and was summarized on slide 7 of the final report (http://sites.google.com/site/ckdbiomarkerscontortium/file-cabinet, the file titled Urine Biospecimen meeting2.ppt). The report contained the following recommendations:

(Summary recommendations of working group) Group 2: Collection, Handling, and Long-Term Storage
Samples should be handled by laboratory staff trained for the activity, with appropriate competency assessment (ISO and CLSI).
Research protocols should match clinical use protocols when possible to minimize errors and speed translation.
Serum and dipotassium EDTA plasma samples should be collected and paired with urine samples.
A separate aliquot of urine should be characterized by multi-parameter dipstick and discarded.
Determining urine creatinine may be useful before storage.
Urine should be kept at 4° C. to 20° C. and centrifuged less than 2 hours after collection.
If not possible, refrigerate, warm and mix, and then centrifuge.
In case of further delay in transport, consider freezing.
Document collection, handling, and transfer steps in detail.
Develop a detailed SOP, including a specific thawing and mixing protocol.
Develop a detailed and standardized SOP for patient procedures and pilot the process for verification.

Note the unqualified recommendation that urine be centrifuged before analysis. This practice will eliminate the ability to accurately assess protein to uromodulin ratios in the samples. These documents make it apparent that analysis of uromodulin is not viewed as highly important for analysis of kidney disease and/or that the ratio of urinary proteins to uromodulin is not understood to be important to diagnosis of kidney disease.

Overall, the experimental results presented in support of this invention show that the excretion of albumin and uromodulin in urine are linked. Disruption of that linkage is the earliest sign of kidney malfunction or loss of kidney volume. Consequently, the AUR presents a substantially improved method for early detection of kidney dysfunction that well precedes the current clinical definition of kidney disease.

Additional details regarding the form of kidney disease may be obtained from the relative value of TUR vs. AUR. These measures were poorly correlated in control groups but highly correlated in groups with elevated AUR or TUR. For example, the Pima Indian cases showed a highly significant correlation between AUR and TUR (R=0.66, p=0.0006). The basis for this difference between controls and cases may offer additional information regarding the type of early kidney disease. For example, albumin and transferrin are filtered by the glomerulus. Free albumin may be completely taken up in the proximal tubules while uptake of free transferrin is incomplete. Elevated release of transferrin into the urine would therefore arise primarily from increased filtration by glomeruli. In contrast, elevated levels of albumin in the urine would indicate incomplete uptake in the proximal tubules. As a result, elevation of only transferrin will suggest glomerular disease while elevation of only albumin will indicate loss of function in the proximal tubules. The Pima Indian cases had elevated TUR and AUR but with greater change of TUR. This suggested declined function of both glomerulus and proximal tubules but with greater impact on the glomerulus. The diabetic group described in FIG. 5 showed greater elevation of AUR than TUR. This suggested greater dysfunction of the proximal tubules.

At more advanced stages of kidney dysfunction, correlation of albumin and transferrin (or AUR and TUR) was always observed. For example, the kidney transplant patients with no apparent organ complication showed elevated AUR (3.67 relative to the standard) and TUR (3.24) with a significant correlation between the two values (R=0.60, p=0.004). The albumin to standard and transferrin to standard were only slightly increased in these subjects, 1.6-fold and 1.22-fold, respectively. However, uromodulin was substantially decreased (0.43 relative to the standard) resulting in a large overall change in AUR and TUR. Loss of the albumin-uromodulin linkage due to incomplete re-uptake would result in similar basis for appearance of both proteins in the urine and a high correlation for albumin and transferrin (or AUR and TUR).

Samples of transplant recipients who were undergoing tests for possible rejection or other problem had been centrifuged, preventing accurate comparison of AUR and TUR. However, elevated albumin relative to the standard (2.20-fold increase) as well as transferrin relative to the standard (2.16-fold increase) were observed. This group showed a very strong correlation between albumin and transferrin in the urine (R=0.85, p=<0.0001). Overall, cases with very large increase of albumin and transferrin (over uromodulin) have broken the albumin-uromodulin linkage and show a high degree of correlation between albumin and transferrin in the urine. At smaller increases in these values, selective changes may provide an indication of the location of decline of function in the kidney.

TABLES 7A-B
Observed vs. theoretical values for deviation from the mean.
A. Protein—protein linkages determined by Observed vs. theoretical
values in 20 thin adults

| Protein to | Observed Delta | Theoretical Delta | |
|---|---|---|---|
| uromodulin | Average (SD) | Average (SD) | p |
| albumin | 0.13 (0.10) | 0.29 (0.17) | 0.00075 |
| kininogen 1 | 0.24 (0.18) | 0.35 (0.22) | 0.08 |
| transferrin | 0.41 (0.38) | 0.43 (0.35) | 0.86 |
| alpha-1-micro- | 0.29 (0.20) | 0.35 (0.20) | 0.29 |
| globulin (Bikunin) | | | |
| Zn-alpha-2- | 0.40 (0.29) | 0.44 (0.28) | 0.72 |
| glycoprotein | | | |
| leucine-rich | 0.46 (0.32) | 0.60 (0.39) | 0.18 |
| alpha-2- | | | |
| glycoprotein | | | |
| epidermal growth | 0.22 (0.13) | 0.25 (0.11) | 0.50 |
| factor | | | |

B. Protein—protein linkages determined by Observed vs. theoretical
sigma/mean values for all control samples (n = 84).

| Protein to | | | |
|---|---|---|---|
| uromodulin | Observed (SD) | Theoretical (SD) | p |
| albumin | 0.18 (0.12) | 0.311 (0.18) | 9.1*10⁻⁰⁷ |
| kinninogen 1 | 0.25 (0.24) | 0.35 (0.23) | 0.009 |
| transferrin | 0.26 (0.24) | 0.35 (0.22) | 0.0095 |
| alpha-1-micro- | 0.39 (0.28) | 0.44 (0.25) | 0.151 |
| globulin (bikunin) | | | |
| alpha-2- | 0.52 (0.42) | 0.56 (0.39) | 0.54 |
| glycoprotein, zinc | | | |
| orosomucoid 1 | 0.54 (0.42) | 0.59 (0.36) | 0.39 |
| precursor | | | |
| leucine-rich | 0.53 (0.36) | 0.39 (0.36) | 0.044 |
| alpha-2- | | | |
| glycoprotein 1 | | | |
| epidermal growth | 0.25 (0.17) | 0.33 (0.19) | 0.014 |
| factor | | | |

### Example 8

### AUR During GFR Test or as a Result of Microalbuminuria

The concept of a stable AUR was also illustrated by AUR before and at the end of a glomerular filtration rate (GFR) determined in a population of adolescents with type 1 diabetes.

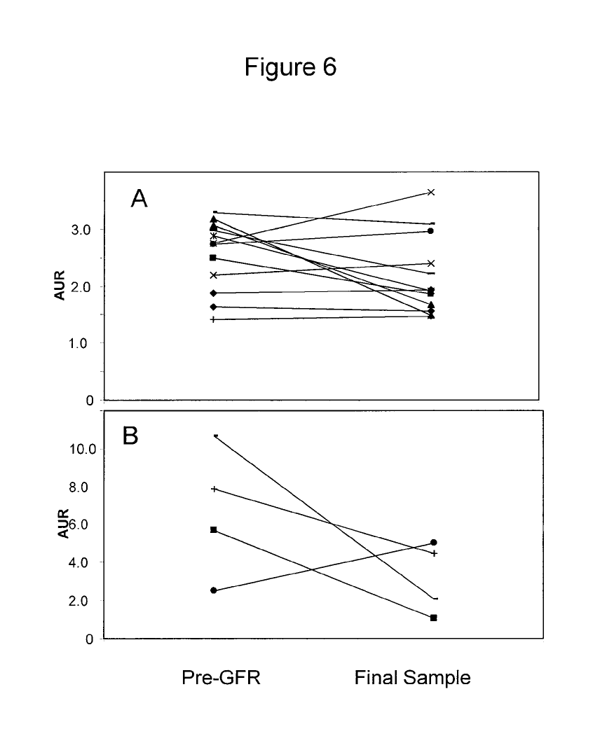

GFR measure involves administration of large volume of liquid to induce urination and excretion of small molecules that have been administered to the test subjects in order to test kidney filtration rates. The individuals in this case all had type 1 diabetes and it is expected that some will eventually advance to kidney disease. The average amount of protein isolated by the standard work-up procedure in the pre-GFR sample was 29.9 micrograms per mL while the protein isolated from the final sample of the GFR test was 6.9 micrograms per mL of urine. Despite more than 4-fold change in urine concentration, the AUR remained constant or even declined slightly in most individuals (FIG. 6A). A small number of individuals had unusually high AUR either before or at the end of the GFR (FIG. 6B). It is possible that these individuals are at greatest risk of developing kidney disease. Long-term follow-up information on these individuals was not available.

Relatively constant AUR but with slight decline in more dilute urine was consistent with findings in other cases described above where more dilute samples correlated with lower AUR values. However, this large change in urine concentration served to emphasize further the relative stability of AUR. Similar results were obtained for TUR.

The correlation of AUR with ACR was determined in 13 subjects with microalbuminuria as defined by an ACR greater than 30 micrograms albumin/mg creatinine. A plot of albumin/uromodulin ratio (relative to the standard sample) vs. ACR gave: slope=0.09, intercept=1.5, R=0.96. The albumin/uromodulin ratio at the ACR defined as the minimum for microalbuminuria, 30 ug of albumin/mg creatinine, was 4.0. An AUR of 4.0 in the different control groups ranged from 7.7 to 8.8 standard deviations above the average. In contrast, the value of 30 micrograms/mg creatinine was 3 SD above the ACR for the Pima Indian study and would be less in control groups from other studies. This evidence shows that the AUR will better detect early changes than the ACR. As in other groups with elevated AUR, transferrin and albumin ratios to the standard correlated extremely well (R=0.96, p<0.0001).

### Example 9

### Prognosis of Chronic Kidney Disease from the Albumin/Uromodulin and Transferrin/Uromodulin Ratios Detected by Antibodies Directed to the Intact Proteins

Albumin and uromodulin in the case and control samples from the Pima Indian study (example 3) were determined by standard ELISA kit assays conducted according to manufacturer's instructions (Bethyl Laboratories, Inc., Montgomery, Tex. and MDBioproducts, Zurich for albumin and uromodulin kits, respectively). Transferrin was also determined by an assay purchased from Bethyl Laboratories, Inc. The values reported are the average of duplicate assays. The assays were conducted on samples that had been exposed to at least 3 freeze-thaw cycles.

The results show that persons who developed proteinuria within 10 years had higher AUR and TUR at baseline (Table 8). The average difference from controls was almost identical to the difference determined at the peptide level by iTRAQ methods as well as to the ACR at baseline. However, the standard deviations for the ELISA assays were very large, eliminating the ability for individual prognosis. The difference was also not significant. However, the results provide proof of principle that antibodies to the intact proteins can detect changes in AUR.

TABLE 8
ELISA assays of samples at baseline.

| Protein or ratio | Cases (SD) | Controls (SD) | Cases/controls | p |
|---|---|---|---|---|
| Albumin ug/mL | 5.5 (5.3) | 3.7 (3.0) | 1.49 | 0.21 |
| | ug/mL | | | |
| Transferrin | 0.185 (0.22) | 0.11 (0.096) | 1.68 | 0.30 |
| Uromodulin | 38.8 (44.9) | 67.1 (80.7) | 0.58 | 0.16 |
| Albumin/ | 0.32 (0.35) | 0.17 (0.24) | 1.86 | 0.12 |
| uromodulin | | | | |
| Transferrin/ | 0.010 (0.020) | 0.0028 (0.0075) | 3.51 | 0.16 |
| Uromodulin | | | | |
| Albumin/urinary | 7.8 (6.8) | 4.4 (6.1) | 1.76 | 0.10 |
| creatinine | | | | |

Albumin was also determined by an ELISA assay that was constructed by standard procedures using antibodies from Bethyl Laboratories. Analysis by antibody-based assays can be improved by addition of agents that dissociate aggregates. In one case, the standard urine sample was concentrated 10-fold by centrifugation of the liquid urine in a Centricon tube. The concentrated sample contained 209 micrograms of BioRad-detected protein per mL. The concentrated sample was diluted 1:500, 1:1000 and 1:2000-fold. A detergent, Tween 20, was added to sample dilution buffer at concentrations 0.05%, 0.1% and 0.2%, 0.5%, 1%, 2%. Table 9 shows the concentrations (in microgram/mL) measured at different concentrations of Tween 20 detergent in the dilution buffer. Values are the concentration of albumin in the 10-fold concentrated urine. From the results, it is apparent that the optimum and most consistent results were obtained at 1.0% Tween 20. Tween 20 at 0.1% and 0.2% gave results similar to those at 0.05%. The concentration, approximately 32 micrograms per mL of the 10× concentrated urine, or 3.2 micrograms per mL of urine, is typical of human urine from healthy individuals. Furthermore, albumin was 15.3% of the BioRad-detected protein in this sample, the same value that was obtained for this standard by the spike-in experiment in example 2. Such excellent agreement of disparate methods of quantification illustrate the necessary accuracy for protein quantification. Similar approaches can be used to optimize antibody assays of transferrin and uromodulin.

TABLE 9

| | 0.05% tween 20 | 0.5% tween 20 | 1% tween 20 | 2% tween 20 |
|---|---|---|---|---|
| 1:500 | 21.9 | 29.3 | 32.7 | 19.7 |
| 1:1000 | 26.3 | 30.6 | 32.5 | 26.6 |
| 1:2000 | 29.8 | 40.1 | 30.7 | 33.5 |

In addition to antibodies directed to the intact proteins, it is possible to generate antibodies that are directed to peptides released by proteolysis of the intact proteins. These peptides may be similar to those used in iTRAQ analysis. Antibody analysis of released peptides will eliminate most of the problems described for analysis of intact proteins.

ELISA Assay of Uromodulin

Materials and procedures: Coating buffer: 0.1 M Carbonate-Bicarbonate, pH 9.6 (Per liter: 5.3 g Na2CO3, 4.2 g NaHCO3, 1 g NaN3). 10×PBS buffer: (Per liter: 80 g NaCl, 2 g KCl, 14.4 g Na2HPO4, 2.4 g KH2PO4, adjust pH to 7.4). Washing buffer: 0.05% Tween 20 in pH 7.4 PBS. Dilution buffer 1: (TEA buffer): 500 mM Tris-HCl, 0.5% Triton X-100, 20 mM EDTA, pH 7.5 (Per 100 ml: 6.05 g Tris, 0.5 g Triton X-100, 20 mM EDTA). Dilution Buffer 2: (same as used for albumin assay) Tris, NaCl, 0.05% tween-20. Blocking buffer: dilution buffer 1 containing 0.25% BSA. Coating antibody: Sheep anti human uromodulin (k90071c, Meridian Life Science Inc, Saco Me.) 1.5 ug/ml in coating buffer. Capture antibody: Sheep anti human uromodulin (k90071c, Meridian Life Science Inc, Saco Me.) conjugate to HRP (701-0000, Novus Biologicals, LLC, Littleton, Colo.). Pipette coating buffer in 96-well plate and wash it. Add 50 μl blocking buffer and incubate at 37° C. for 30 min and wash. Uromodulin standard is dissolved in blocking buffer and added into plate at 640, 320, 160, 80, 40, 20, 10 ng/ml and incubated at room temperature for 2 h. The Capture antibody was diluted in dilution buffer to 2 to 4 ug/ml. Add 50 μl to each well and incubate for 2 h at room temperature. Add 50 μl TMB to each well, incubate at room temp for about 15 min.

Assay of the standard sample. The standard urine sample was concentrated 10× and analyzed for uromodulin by ELISA. BioRad-detected protein concentration of the concentrated sample was 209 ug/mL. A criterion for a satisfactory assay was a constant uromodulin determination at different dilutions of the sample. This established that the unknown sample followed the same standard curve as pure uromodulin. Table 9A below shows greatest consistency with sample dilution into buffer 1. The ratio of uromodulin to BioRad-detected protein in the standard urine sample was 214/209=1.02, very similar to the ratio of 0.95 obtained by the spike-in experiment that was quantified by iTRAQ analysis in example 2.

TABLE 9A
Uromodulin of a standard sample in micrograms
per mL of 10X concentrated urine.

| Standard | Di- | Di- | Di- | Di- | | iTRAQ |
|---|---|---|---|---|---|---|
| sample 10X | luted | luted | luted | luted | Ave | spike-in |
| concentrated | 1:500 | 1:1000 | 1:2000 | 1:4000 | (SD) | result |
| Buffer 1 | 214 | 234 | 203 | 207 | 214 (14) | 190 |
| Tris, 0.5% | | | | | | |
| triton X100, | | | | | | |
| 20 mM EDTA | | | | | | |
| Buffer 2 | 213 | 274 | 297 | 270 | 263 (36) | 190 |
| Tris, NaCl, | | | | | | |
| 0.05% tween- | | | | | | |
| 20 | | | | | | |

A number of experimental samples from the Pima Indian study proved problematic for uromodulin assay by this method. In fact, the majority of samples did not meet the criterion of constant protein detected at different dilutions of the sample. These samples had been stored for over 15 years. They had also been subjected to several freeze-thaw cycles. The results for the standard sample show that antibody assays can be effective for quantification of uromodulin, but careful sample handling is needed. Long term storage and multiple freeze-thaw cycles may prevent accurate quantification by assays with antibodies directed to intact proteins.

### Example 10

### Protein Quantification at the Peptide Level: MALDI-TOF Mass Spectrometry

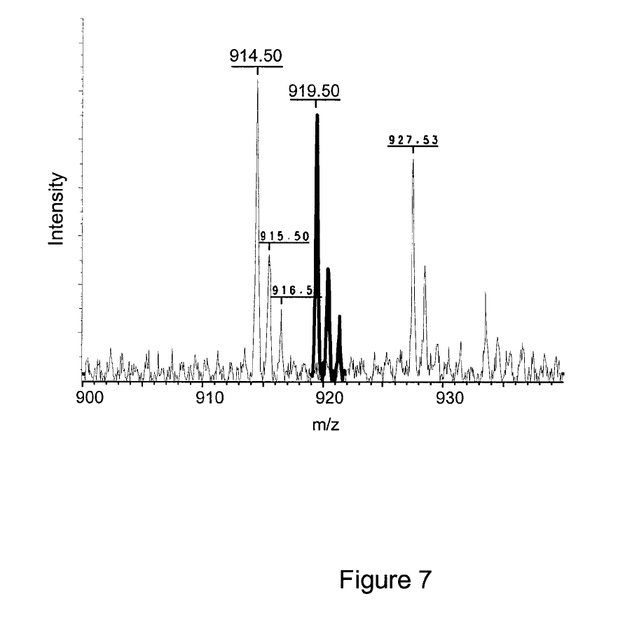

In some cases, peptides can be identified and quantified by MS analysis in the MALDI-TOF mass spectrometer. Despite a complex sample, some peptides are very intense in the MALDI-TOF mass spectrometer and appear far above background. In this case, a simple instrument and procedure can be conducted. After urine protein digestion with an appropriate protease enzyme, a heavy atom peptide can be added and the sample extracted by a ZipTip that isolates the peptides from contaminating materials. The peptides are applied to a MALDI target along with a matrix material. After drying, the sample is subjected to laser shots that liberate the peptides that are then detected by a Time of Flight instrument. A uromodulin peptide (FSVQMFR (SEQ ID NO:1)) at m/z=914.46 was well separated from other peaks found in urine (FIG. 7) and was very intense. Addition of a synthetic peptide containing 5 ¹³C atoms to the sample before analysis will produce a new component that is illustrated by the bold line in FIG. 7. The peptide of the sample can be quantified by comparison of its intensity or area under the curve to that of the heavy atom, spike-in peptide.

### Example 11

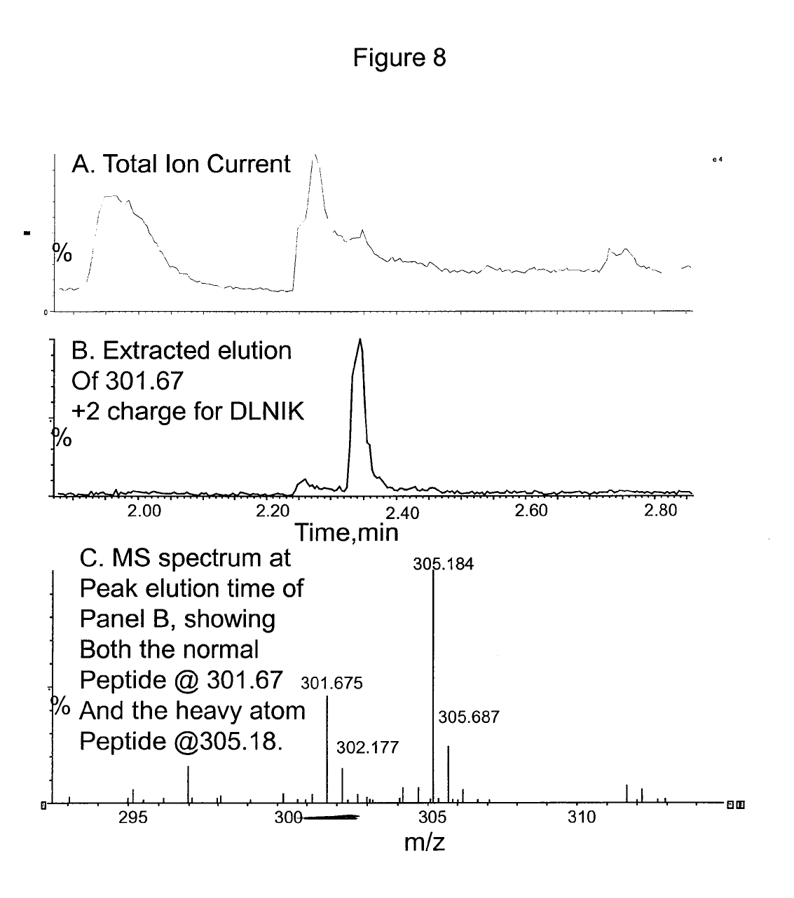

### Analysis of Proteins at the Peptide Level by Mass Spectrometry Methods: Direct Quantification of Peptides Released by Protein Digest (FIG. 8)

Direct quantification of peptides after protease digestion can be carried out without fragmentation in the mass spectrometer. This method also relies on heavy atom peptides of known concentration that are added to the sample. FIG. 8 shows results for one peptide marker of uromodulin (DLNIK (SEQ ID NO:2)). The standard urine sample was concentrated 10-fold and dialyzed against 20 mM NH₄HCO₃. The disulfide bonds were reduced and the free sulfhydryls were alkylated with iodoacetamide as described for the iTRAQ analysis, above. The protein was digested with trypsin and the digest applied directly to a reverse phase 18C column that was eluted with a gradient of acetonitrile. The total ion current (FIG. 8A) was detected by a Waters Premier XE ESI-TOF mass spectrometer. Isolation of the elution profile for the m/z=301.67 signal gave a single intense peak (FIG. 8B). The MS spectrum at the center of this peak (FIG. 8C) revealed the most intense peak was the +2 charge state of the peptide (m/z=301.67). The +1 charge state was also abundant at m/z=602.35. This sample had been spiked with a heavy atom peptide containing ¹³C atoms. The heavy atom peptide was observed at m/z=305.18. The isotopes for each peptide occur at 0.5 mass unit intervals, in agreement with a +2 charge state. The heavy atom peptide had been added at 4 micrograms per mL. The extracted ion current for both the normal and heavy atom peptides were integrated. The result indicated a concentration of 251.99 micrograms of uromodulin polypeptide chain per mL. The peaks for the +1 charge states for the normal and heavy atom peptide were also integrated and compared. The same calculations were conducted for the +1 and +2 charge states for two peptides. The overall average for all values was 273.4+/−19.5 (CV=7%). This approach was also applied to peptides of albumin for which heavy atom peptides had been synthesized. These peptides provided a concentration of 37.4+/−1.48 micrograms albumin per mL. These values and especially the ratio of albumin to uromodulin were very similar to values obtained by the spike-in experiment that was analyzed by iTRAQ and to the ELISA assay that used antibodies to the intact proteins. An alternative to the method of FIG. 8 is analysis of product ions of the parent peptide. The latter type of analysis is referred to as MRM and the general principles of this method are illustrated below.

Overall, the ratio of proteins in the urine can be determined by several different approaches. The greatest concern is for antibody based assays where aged samples or repeated freeze-thaw cycles can lead to inaccurate values.

### Example 12

### Analysis of Proteins at the Peptide Level by Mass Spectrometry Methods: MRM Analysis

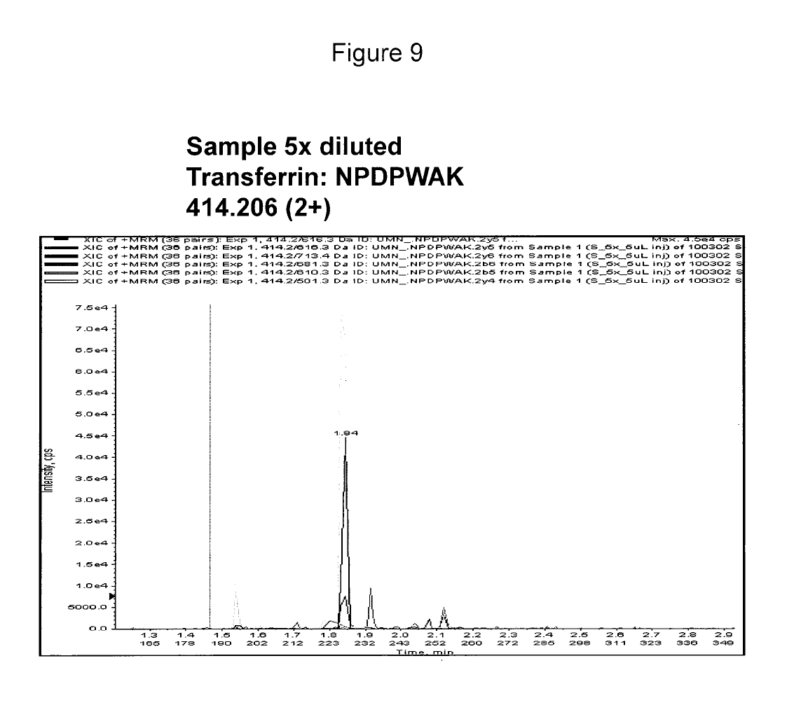

Multiple reaction monitoring (MRM) is a method known in the art for quantification of peptides and other small molecules. The MRM method consists of protein digestion as described for iTRAQ analysis and chromatographic separation of the peptides. The mass spectrometer (most often a triple quadrupole mass spectrometer) is programmed to analyze the peptide masses that are programmed into the instrument. The instrument detects product ions from the target peptides and quantifies the peptide relative to an identical peptide that has been added to the sample but that contains heavy atoms that result in separation of the spike-in peptide from the sample peptide by m/z values. The protein in the target sample is then quantified by the intensity or area under the curve of the product ions of the sample peptide relative to those of a spike-in peptide of known concentration. An example of this is shown in FIG. 9. This peptide of transferrin was identified by mass spectrometry analysis and eluted at 1.84 minutes from the column. It was observed as a +2 charge state at m/z=414.2. Important product ions that can be used to quantify the peptide are shown at m/z=501.3, 616.3 and 713.4 (+1 charge state in all cases). In the experimental application of this approach, the sample can contain an added heavy atom peptide of the same sequence, e.g., with 5 C-13 atoms per peptide. In this case, the parent ion will be at m/z=416.7 and the corresponding product ions at 506.3, 621.3 and 718.4. Quantification of the peptide of the sample will be achieved by comparison to the intensity or area under the curve for the added peptide.

Other methods for relative MRM analysis by peptide labeling procedures have been described by commercial firms such as Applied Biosystems, Inc. One example involves label of the amino terminal group of each peptide of two samples with a different reagent, similar to the iTRAQ reagent. The peptides are then analyzed and the level of a peptide in the experimental sample is obtained by comparison to the peptide in the standard sample, using methods similar to those outlined in iTRAQ analysis. The standard can be a biological sample, a pure protein or peptides corresponding to the targets of analysis.

In general, the MRM method and variations on its application are known in the art. It is expected that additional variations on this method will be developed and can be used in the manner outlined in this example.

### Example 13

### Tryptic or Other Protease Digestion of Urine Proteins

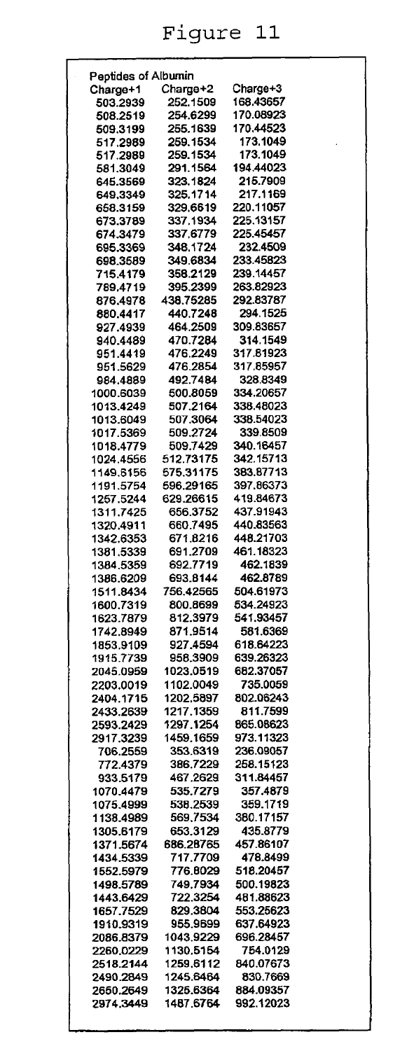

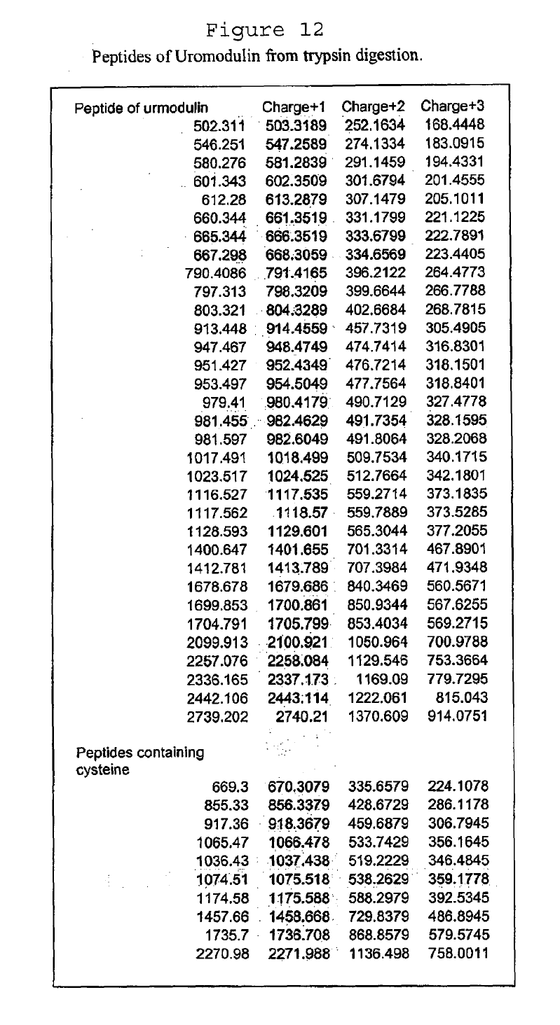

The methods of iTRAQ, MALDI-TOF, ESI-TOF or MRM analysis can be applied to any appropriate peptide of a target protein. Most commonly, peptides are produced by trypsin digestion by methods outlined above. Theoretical peptides can be obtained by theoretical digestion of proteins by standard programs such as those provided by the Expasy web site. A list of m/z values for peptides of >500 Dalton mass released by trypsin digestion of albumin and uromodulin are given in FIGS. 11 and 12, respectively. Peptides for analysis can include any of those listed. In some cases, smaller peptides can be used. In addition, some peptides may be modified by oxidation or other known post-translational modification. The m/z values for these peptides can also be used for quantification by the methods outlined above.

In addition to trypsin, many other protease enzymes of known specificity can be used. The Expasy Website can be consulted for a list of current proteases and software can be used to provide a list of theoretical peptides from each that might be used for analysis.

### Example 14

### Glycoprotein Analysis and Use in Prognosis

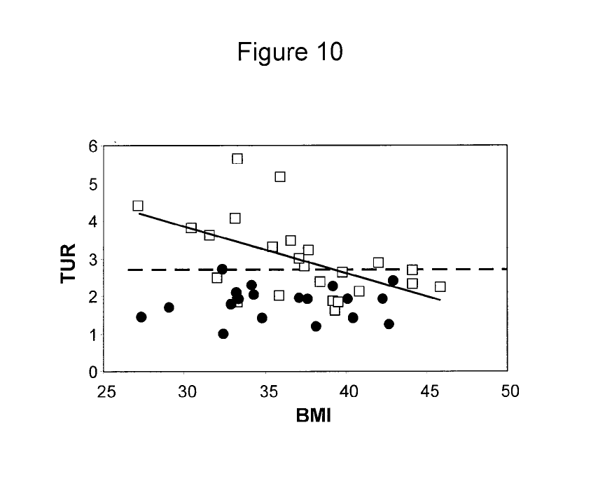

A surprising finding was a correlation between TUR and BMI for cases but not controls of the Pima Indian study (FIG. 10, Table 10). In contrast, the correlations between AUR or ACR with BMI were not significant. A feature that distinguishes transferrin from albumin is the presence of two N-linked complex carbohydrate chains on transferrin. In fact, most of the glycoproteins observed in this study showed similar trends as that for transferrin (Table 10). Glycoprotein concentration appeared to be linked to BMI in cases but not controls. This correlation can be used to further enhance prognosis of cases that progress to kidney disease when applied in a manner that accounts for BMI. Persons of lower BMI had better prognosis by these protein ratios.

No correlations were found between these glycoproteins of the urine and either age or duration of diabetes.

TABLE 10
Plots of protein/uromodulin ratio vs. BMI with correlation coefficients.

| | A. Cases: Slope | B. Controls: Slope | C. Cases, slope |
|---|---|---|---|
| | for Protein/uromodulin | for Protein/uromodulin | for protein alone |
| Protein | vs. BMI (R, p) | vs. BMI (R, p) | vs. BMI. Slope (R, p) |
| Albumin | −0.06 (0.34, 0.12) | 0.013 (0.16, 0.46) | −0.023 (0.32, 0.14) |
| Transferrin | −0.12 (0.53, 0.01) | 0.005 (0.05, 0.82) | −0.050 (0.43, 0.04) |
| Alpha-2-GP, Zn | −0.19 (0.37, 0.08) | −0.019 (0.04, 0.86) | −0.090 (0.47, 0.02) |
| Orosomucoid | −0.17 (0.57, 0.004) | −0.019 (0.27, 0.21) | −0.080 (0.58, 0.004) |
| Leucine-Rich | −0.15 (0.40, 0.06) | −0.050 (0.32, 0.14) | −0.06 (0.39, 0.07) |
| Alpha-2-GP | | | |
| Uromodulin | NA | NA | 0.0011 (0.034, 0.88) |

### Example 15

### Disease Diagnosis by Glycopeptides Released after Protease Digestion

Many of the proteins described herein contain carbohydrate chains. As shown in example 14, the overall presence of glycoproteins can be linked to BMI in disease states. Another approach to analysis of glycoproteins is the determination of glycosylation at specific sites. Glycoproteins are known to be heterogeneous and to contain incomplete glycosylation. With two carbohydrate chains, transferrin is known to exist as polypeptides containing 0, 1 and 2 carbohydrate chains. The current methods allow rapid analysis of urine glycoproteins to determine their relative glycosylation states. Among the proteins described in this document, glycosylation states of tryptic peptides are given in Table 11. The table also includes the result of data search for the unglycosylated peptide. In those cases examined, “No” indicates that the unglycosylated peptide was not detected while “Yes” indicates that the peptide was found. If no response is listed in Table 11, the presence of the peptide was not examined. This analysis was conducted on urine of a healthy individual. Consequently, it is apparent that at least several peptides with under-glycosylation can be used for this analysis. Individuals with certain disease states will display either greater or lesser levels of the unglycosylated peptides relative to the corresponding glycosylated peptides.

The optimum peptides for analysis will have masses that provide +1, +2, +3 or +4 charge states at m/z values less than 2000. Peptides with only one glycosylation site are also preferred. The level of glycosylation of each site can be determined by the amount of the unglycosylated peptide determined by any of the methods discussed herein or available to the art, relative to peptides of the same protein that are not targets of glycosylation during biosynthesis. It is possible to detect the percentage of each site that is glycosylated. Glycosylation state can then be correlated with disease. Glycosylation of uromodulin can be correlated with kidney disease. Glycosylation of the other proteins can be correlated with disease of the organ of synthesis, most commonly the liver. However, other glycoproteins of the urine such as kinninogen 1 arise partly from the kidney and glycosylation states may report on kidney disease as well. Any glycoprotein of the urine can be used for this type of analysis. Proteases other than trypsin can be used to generate the peptides from the intact proteins.

TABLE 11
Glycopeptides of common urine proteins.
Glycosylation sites are indicated by larger bold type.

| | | | Intensity of |
|---|---|---|---|
| | | | unglycosylated |
| | | | peptide found |
| | | Peptide Mass | in ESI-TOF |
| | | (+Carboxymethylamido | mass |
| Protein | Tryptic peptide sequence | derivative) | spectrometer |
| Uromodulin | | | |
| 31-99 (3 | [symbol] | 7796.05 | No |
| chains) | TCQEGFTGDGLTCVDLDECA | | |
| | [symbol] | | |
| | SCVCPEGFR (SEQ ID NO: 3) | | |
| 266-307 | [symbol] | 4847.94 | No |
| | YCTDPSSVEGTCEECSIDED CK | | |
| | (SEQ ID NO: 4) | | |
| 223-245 | CNTAAPMWLNGTHPSSDEGI | 2499.13 | No |
| | VSR (SEQ ID NO: 5) | | |
| 396-415 | [symbol] | 2336.17 | No |
| | (SEQ ID NO: 6) | | |
| 319-332 | [symbol] | No | |
| Transferrin | | | |
| 47-60 | [symbol] | 1414.72 | |
| 622-642 | [symbol] | 2514.12 | |
| | (SEQ ID NO: 9) | | |
| 421-433 | [symbol] | 1475.75 | |
| | (SEQ ID NO: 10) | | |
| Orosomucoid | | | |
| 19-42 | [symbol] | 2575.41 | |
| | ITG (SEQ ID NO: 11) | | |
| 52-57 | [symbol] (SEQ ID NO: 12) | 795.35 | Yes |
| 58-73 | [symbol] | 1918.95 | Yes |
| | (SEQ ID NO: 13) | | |
| 87-101 | [symbol] (SEQ ID | 1914.90 | |
| | NO: 14) | | |
| 102-108 | [symbol] (SEQ ID NO: 15) | 775.39 | Yes |
| Alpha-2- | | | |
| glycoprotein- | | | |
| Zinc | | | |
| 100-117-2 | [symbol] | 2064.96 | |
| chains | (SEQ ID NO: 16) | | |
| 118-126 | [symbol] (SEQ ID NO: 17) | 1137.49 | Yes at +2 |
| | | | charge state |
| 249-295 | [symbol] | 5096.46 | |
| | PQDTAPYSCHVQHSSLAQPL | | |
| | VVPWEAS (SEQ ID NO: 18) | | |
| Leucine-rich | | | |
| alpha-2- | | | |
| glycoprotein | | | |
| 36-41 | [symbol] (SEQ ID NO: 19) | 643.40 | |
| 48-93 | SDHGSSISCQPPAEIPGYLP | 4898.44 | |
| | [symbol] | | |
| | LQGASK (SEQ ID NO: 20) | | |
| 179-191 | [symbol] (SEQ ID | 1423.86 | |
| | NO: 21) | | |
| 261-291 | [symbol] | 3456.65 | |
| | ASLGQPNWDMR (SEQ ID | | |
| | NO: 22) | | |
| 321-328 | [symbol] (SEQ ID NO: 23) | 997.44 | |

### Example 16

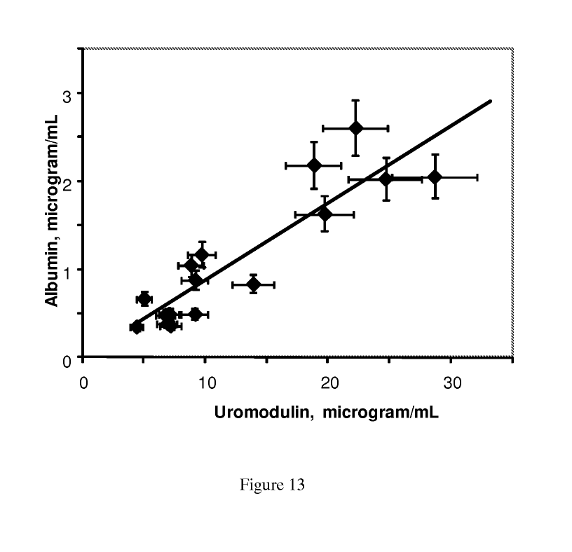

Use of heavy atom peptide spike-in experiments is illustrated by studies of healthy control subjects. FIG. 13 shows the levels of albumin and uromodulin determined by spike-in of three peptides per protein by the procedure outlined above. The correlation coefficient for these proteins in this determination was 0.90 and the coefficient of variation from the mean was 31%. This precision approached that obtained by iTRAQ technology. These samples had been subjected to two freeze-thaw cycles before this assay was conducted. In comparisons of other sample groups, it was found that two freeze-thaw cycles altered the ratio of albumin to uromodulin due to greater decline of the less abundant protein, albumin.

### BIBLIOGRAPHY

1. Han, C. L., et al. (2008) Mol Cell Proteomics 7, 1983-1997.
2. Jantos-Siwy, J., et al. (2009) J Proteome Res 8, 268-281.
3. Nickolas, T. L., et al. 2008) Curr Opin Nephrol Hypertens 17, 127-132.
4. Perco, P., et al. (2006) Eur J Clin Invest 36, 753-763.
5. Stenvinkel, P., et al. (2008) Clin J Am Soc Nephrol 3, 505-521.
6. Thongboonkerd, V. (2008) Contrib Nephrol 160, 142-158.
7. Zurbig, P., et al. (2009) Proteomics 9, 2108-2117.
8. Rossing, K., et al. (2008) J Am Soc Nephrol 19, 1283-1290.
9. Otu, H. et al. (2007) Diabetes Care 30, 638-643.
10. Akkina, S. K., et al. (2009) J Proteome Res 8, 94-103.
11. Howard, R. L., et al. (1991) Kidney Int 40, 923-926.
12. Jain, S., et al. (2005) J Assoc Physicians India 53, 513-520.
13. Jirka, J., et al. (1976) Biomedicine 25, 246-248.
14. Christiansen, M. S., et al. (2002) Diabetologia 45, 115-120.
15. Sviridov, D., and Hortin, G. L. (2009) Clin Chim Acta.
16. Lynn, K. L., et al. (1982) Clin Sci (Lond) 62, 21-26.
17. Kobayashi, K., and Fukuoka, S. (2001) Arch Biochem Biophys 388, 113-120.
18. Dong, M. Q., et al. (2007) Science 317, 660-663.
19. Kuhn, E., et al. (2004) Proteomics 4, 1175-1186.
20. Chalcraft, K. R., and Britz-McKibbin, P. (2009) Anal Chem 81, 307-314.
21. Bleyer A J, et al. (2004) Kidney Int 66:974-977.
22. Kottgen A, et al. (2010) J Am Soc Nephrol. 2010 February; 21(2):337-44.
23. ILIR SEJDIU & OLE TORFFVIT, (2008) Scandinavian Journal of Urology and Nephrology, 42:168_{—}174.
24. Pfleiderer S, et al. (1993) Clin Invest 71:/972_{—}7.

25. Sinikka Prajczer, et al. (2010) Nephrology Dialysis Transplantation, NDT Advance Access published online on Jan. 14, 2010 doi:10.1093/ndt/gfp748.
26. Olivier Devuyst, et al. (2005) Nephrology Dialysis Transplantation 20(7):1290-1294.
27. S. Gonzalez, L. Vargas, (2001) Horm Metab Res 33: 84±88.
28. ZELLER A. et al. (2005) Journal of human hypertension vol. 19, no3, pp. 205-209.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

1. A method for diagnosing kidney disease from a urine sample comprising determining the ratio of a first protein to a second protein present in said urine sample from a subject, wherein the ratio indicates the presence of kidney disease in the subject.

2. A method for screening a subject at risk for developing kidney disease comprising determining the ratio of a first protein to a second protein present in urine from said subject, wherein the ratio indicates that the subject is at risk for developing kidney disease.

3. A method for identifying and treating kidney disease in a subject comprising determining the ratio of a first protein to a second protein present in urine from said subject, wherein the ratio indicates the subject has kidney disease, and administering a treatment for kidney disease to the subject.

4. A method for determining whether a subject has kidney disease comprising determining the ratio of a first protein to a second protein present in urine from said subject, wherein the ratio indicates that the subject has developed kidney disease.

5. This method of claim 1, wherein the sample is obtained from a subject at risk for developing kidney disease.

6. The method of claim 1, wherein a urine sample is obtained from a subject.

7. The method of claim 1, wherein the subject has a history of diabetes, hypertension (high blood pressure), obesity, sickle cell disease, lupus erythematosus, atherosclerosis, glomerulonephritis, bladder outlet obstruction, overexposure to toxins and to some medications, a family history of kidney disease including polycystic kidney disease, is over the age of 60 and/or is a member of one of the following ethnic groups American, African American Indian, Hispanic, Asian American, or Pacific Islander.

8. The method of claim 3, wherein the treatment comprises surgery, chemotherapy, radiation therapy, dietary restrictions, treatment of high blood pressure, treatment of diabetes, weight management, smoking cessation, treatment of high cholesterol and/or other lipid levels, kidney transplant, administration of erythropoietin, diuretics, vitamin D, or phosphate binder or a combination thereof.

9. The method of claim 1, wherein the second protein is uromodulin.

10. The method of claim 1, wherein the first protein is selected from the group consisting of albumin, transferrin, alpha-2-glycoprotein-Zinc, orosomucoid, or leucine-rich alpha-2-glycoprotein.

11. The method of claim 1, wherein the ratio of the first protein to uromodulin that is characteristic of early stage kidney disease is at least about 2 standard deviations above the average for a control population.

12. The method of claim 1, wherein the ratio for albumin to uromodulin is greater than about 0.30 (w/w).

13. The method of claim 1, wherein the urine protein ratio is combined with at least one other indicator of kidney disease to diagnose development of kidney disease.

14. The method of claim 13, wherein the other indicator comprises fasting blood glucose, glucose tolerance test outcome, hemoglobin A1C levels, or blood pressure.

15. The method of claim 1, wherein the urine proteins are detected by an antibody-based assay.

16. The method of claim 15, wherein the antibodies are directed to intact protein.

17. The method of claim 1, wherein the urine proteins have been digested with a protease to yield peptides.

18. The method of claim 17, wherein the peptides are detected by antibody methods.

19. The method of claim 17, wherein the peptides are detected by mass spectrometry methods.

20. A method for diagnosing disease comprising determining the glycosylation state of urinary peptides that are present in urine or released from urinary proteins by protease digestion, wherein the method comprises measuring the amount of the non-glycosylated form of a putative glycosylated peptide and comparing that to the amount of a peptide of the same protein that is not a target for glycosylation, wherein greater or lesser levels of the unglycosylated peptides compared to a healthy control indicates disease.

21. The method of claim 20, wherein the origin of the protein is liver and the disease diagnosis is liver disease.

22. The method of claim 20, wherein the origin of the protein is kidney and the disease diagnosis is kidney disease.

23. The method of claim 20, wherein the peptide is alpha-2-glycoprotein-Zinc, orosomucoid 1 or 2, or leucine-rich alpha-2-glycoprotein.


SEQUENCE LISTING (24 sequences)

SEQ ID NO: 1 - protein, 7 aa, Homo sapiens
FSVQMFR

SEQ ID NO: 2 - protein, 5 aa, Homo sapiens
DLNIK

SEQ ID NO: 3 - protein, 69 aa, Homo sapiens
WCSECHSNATCTEDEAVTTCTCQEGFTGDGLTCVDLDECAIPGAHNCSANSSCVNTPGSFSCVCPEGFR

SEQ ID NO: 4 - protein, 42 aa, Homo sapiens
ACAGGYYVYNLTAPPECHLAYCTDPSSVEGTCEECSIDEDCK

SEQ ID NO: 5 - protein, 23 aa, Homo sapiens
CNTAAPMWLNGTHPSSDEGIVSR

SEQ ID NO: 6 - protein, 20 aa, Homo sapiens
NETHATYSNTLYLADEIIIR

SEQ ID NO: 7 - protein, 14 aa, Homo sapiens
QDFNITDISLLEHR

SEQ ID NO: 8 - protein, 14 aa, Homo sapiens
SVIPSDGPSVACVK

SEQ ID NO: 9 - protein, 21 aa, Homo sapiens
QQQHLFGSNVTDCSGNFCLFR

SEQ ID NO: 10 - protein, 13 aa, Homo sapiens
CGLVPVLAENYNK

SEQ ID NO: 11 - protein, 23 aa, Homo sapiens
QIPLCANLVPVPITNATLDQITG

SEQ ID NO: 12 - protein, 6 aa, Homo sapiens
NEEYNK

SEQ ID NO: 13 - protein, 16 aa, Homo sapiens
SVQEIQATFFYFTPNK

SEQ ID NO: 14 - protein, 15 aa, Homo sapiens
QDQCIYNTTYLNVQR

SEQ ID NO: 15 - protein, 7 aa, Homo sapiens
ENGTISR

SEQ ID NO: 16 - protein, 18 aa, Homo sapiens
DIVEYYNDSNGSHVLQGR

SEQ ID NO: 17 - protein, 9 aa, Homo sapiens
FGCEIENNR

SEQ ID NO: 18 - protein, 47 aa, Homo sapiens
GDVLHNGNGTYQSWVVVAVPPQDTAPYSCHVQHSSLAQPLVVPWEAS

SEQ ID NO: 19 - protein, 6 aa, Homo sapiens
VTLSPK

SEQ ID NO: 20 - protein, 46 aa, Homo sapiens
SDHGSSISCQPPAEIPGYLPADTVHLAVEFFNLTHLPANLLQGASK

SEQ ID NO: 21 - protein, 13 aa, Homo sapiens
LPPGLLANFTLLR

SEQ ID NO: 22 - protein, 31 aa, Homo sapiens
QLDMLDLSNNSLASVPEGLWASLGQPNWDMR

SEQ ID NO: 23 - protein, 8 aa, Homo sapiens
MFSQNDTR

SEQ ID NO: 24 - protein, 7 aa, Homo sapiens
NPDPWAK